(19) United States
(12) Patent Application Publication
(10) Pub. No.: US 2018/0141965 A1
(43) Pub. Date: May 24, 2018

(54) AUTOTAXIN INHIBITORS AND USES THEREOF

(71) Applicant: NATIONAL AND KAPODISTRIAN UNIVERSITY OF ATHENS, ATHENS (GR)

(72) Inventor: GEORGE KOKOTOS, ATHENS (GR)

(73) Assignee: NATIONAL AND KAPODISTRIAN UNIVERSITY OF ATHENS, ATHENS (GR)

(21) Appl. No.: 15/575,035

(22) PCT Filed: May 18, 2016
(86) PCT No.: PCT/EP2016/000805

§ 371 Date: Nov. 17, 2017

(30) Foreign Application Priority Data
May 18, 2015 (EP) PCT/EP2015/001013

(51) Int. Cl. C07F 9/48; C07C 335/08; C07C 327/42; C07C 259/06; C07C 237/22; C07F 9/40; A61P 35/00; A61P 3/04; A61P 29/00; A61P 37/00

(52) U.S. Cl. CPC C07F 9/4816; C07C 335/08; C07C 327/42; C07C 259/06; A61P 37/00; C07F 9/4006; A61P 35/00; A61P 3/04; A61P 29/00; C07C 237/22

(57) ABSTRACT

Novel inhibitors of the enzyme autotaxin are described. The inhibitors contain one or two zinc-binding groups at the appropriate distance, Also described are uses thereof, such as for the inhibition of autotaxin activity and the treatment of various conditions (e.g., inflammatory conditions, cancer, obesity, autoimmune diseases).

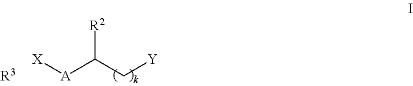

Novel inhibitors of the enzyme autotaxin are described. The inhibitors contain one or two zinc-binding groups at the appropriate distance. Also described are their uses, such as for the inhibition of autotaxin activity and the treatment of various conditions (e.g., inflammatory conditions, cancer, obesity, autoimmune diseases).

### BACKGROUND OF THE INVENTION

Autotaxin (ATX) (also known as ectonucleotide pyrophosphatase/phosphodiesterase 2, Enpp2 or NPP2) is a member of the nucleotide pyrophosphatase/phosphodiesterase family of ectoenzymes, that hydrolyze phosphodiester bonds of various nucleotides and nucleotide derivatives. ATX is a secreted enzyme that possesses lysophopho-lipase D activity and catalyzes the hydrolysis of lysophosphatidylcholine (LPC) to lysophosphatidic acid (LPA) and choline.

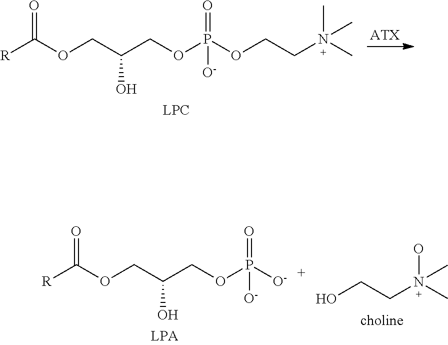

The product of this enzymatic activity, LPA, is a bioactive lipid mediator that binds to specific G-protein-coupled receptors (LPA₁₋₆ in mammals) and activates various signal transduction pathways including the migration, proliferation, and survival of various cell types. ATX is the main provider of LPA in serum. ATX and LPA have been shown to be involved in both pathological and physiological states including tumor progression and metastasis, neuropathy pain, atherosclerosis, arthritis, lung fibrosis, liver fibrosis, brain development, nervous system development, and chronic inflammation disorders. A number of recent review articles summarize the role of ATX in various diseases (see, Barbayianni E. et al, “Autotaxin, a secreted lysophos-pholipase D, as a promising therapeutic target in chronic inflammation and cancer”, Prog. Lipid Res. 2015, 58, 76-96; Benesch M. G. et al, “Autotaxin in the crosshairs: Taking aim at cancer and other inflammatory conditions”, FEBS Lett. 2014, 588(16), 2712-2727; Knowlden S. et al, “The autotaxin-LPA axis emerges as a novel regulator of lymphocyte homing and inflammation”, J. Immunol. 2014, 192(3), 851-7; Sevastou I. et al, “Lysoglycerophospholipids in chronic inflammatory disorders: the PLA(2)/LPC and ATX/LPA axes”, Biochim. Biophys. Acta—Molecular and Cell Biology of Lipids, 2013, 1831, 42-60; Gotoh M. et al. “Controlling cancer through the autotaxin-lysophosphatidic acid receptor axis”, Biochem. Soc. Trans. 2012, 40, 31-36; Houben A. J. et al. “Autotaxin and LPA receptor signaling in cancer”, Cancer Metastasis Rev. 2011, 30, 557-565).

The implications of ATX in human diseases make this enzyme an attractive target for the discovery of novel drug therapies. A variety of synthetic ATX inhibitors have been developed during the last years. Their structures, syntheses, uses and applications are summarized in recent review articles (see, Barbayianni E. et al, “Autotaxin, a secreted lysophospholipase D, as a promising therapeutic target in chronic inflammation and cancer”, Prog. Lipid Res. 2015, 58, 76-96; Barbayianni E. et al, “Autotaxin inhibitors: a patent review”, Expert Opin. Ther. Pat. 2013, 23, 1123-1132; Albers H. M. et al, “Chemical evolution of autotaxin inhibitors”, Chem. Rev. 2012, 112, 2593-2603; Parrill A. L. et al, “Autotaxin inhibitors: a perspective on initial medicinal chemistry efforts”. Expert Opin. Ther. Pat. 2010, 20, 1619-1625).

### SUMMARY OF THE INVENTION

The present invention relates to compounds containing one or two-zinc binding groups as well as salts, and derivatives thereof, and compositions containing them. The invention further relates to uses of such compounds, salts, derivatives and compositions, such as for the inhibition of autotaxin and/or the treatment of various conditions (e.g., inflammatory conditions, cancer, obesity, autoimmune diseases).

In a first aspect, the present invention provides compounds of formula I

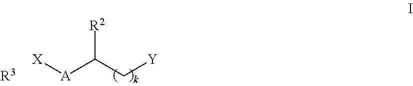

wherein:

A represents

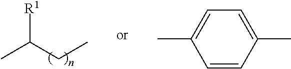

X and Y are groups able to form complexes with Zn²⁺ ion, wherein
Y:

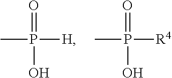

R⁴: lower alkyl, lower alkoxy

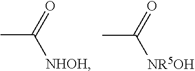

R⁵: phenyl, benzyl, lower alkyl

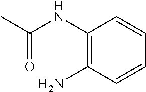

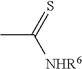

R⁶: lower alkyl, CH₂CH₂NH₂
X:

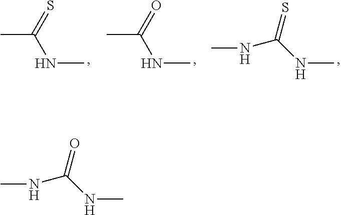

R¹, R² is independently selected from H, lower alkyl, COOR⁷, CONHR⁷, CSNHR⁷, wherein R⁷ is a lower alkyl group.

R³: C₆-C₁₆ (alkyl, alkenyl, alkynyl) or a group selected from

R⁸: aryl, heteroaryl

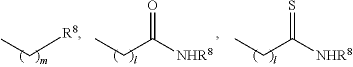

k=0-2

m, l=3-8

n=0-3

or isomers, enantiomeric forms, pharmacologically acceptable salts, or prodrugs thereof.

The applicant has found that there is an essential pharmacophore in the presence of two zinc-binding groups within a distance of 3 to 5 carbons.

In another aspect, the present invention provides a composition comprising a compound of formula I and a pharmaceutically acceptable carrier or excipient.

In another aspect, the present invention provides the above-mentioned compound of formula I for use as a medicament.

In another aspect, the present invention provides a method for inhibiting ATX activity in a system (e.g., a cell-free system), cell or subject, said method comprising contacting said system or cell with, or administering to said subject, an effective amount of the above-mentioned compound of formula I or composition comprising a compound of formula I.

In another aspect, the present invention provides a method for preventing and/or treating an inflammatory disease or condition in a subject, said method comprising administering to said subject a therapeutically-effective amount of a compound of formula I.

In another aspect, the present invention provides the use of the above-mentioned compound of formula I, or composition comprising a compound of formula I, for the preparation of a medicament for inhibiting ATX activity in a system (e.g., a cell-free system), cell or subject.

In another aspect, the present invention provides a compound of formula I, or a composition comprising a compound of formula I, for use in the prevention and/or treatment of an inflammatory disease or condition or for use in the prevention and/or treatment of cancer.

In another aspect, the present invention provides a compound of formula I, or a composition comprising a compound of formula I, for use in the prevention and/or treatment of an inflammatory disease.

In another aspect, the present invention provides a compound of formula I, or a composition comprising a compound of formula I, for use in the prevention and/or treatment of obesity.

In another aspect, the present invention provides a compound of formula I, or a composition comprising a compound of formula I, for use in the prevention and/or treatment of autoimmune diseases.

In another aspect, the present invention provides a process for preparing a compound of formula I, comprises a step of:

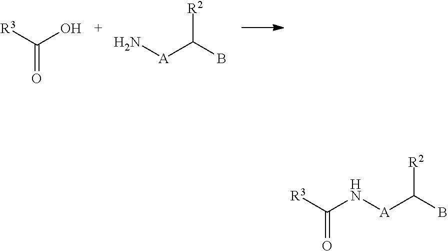

wherein
A represents

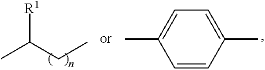

wherein R¹, R² are independently selected from H, lower alkyl, COOR⁷, CONHR⁷, CSNHR⁷, wherein R⁷ is a lower alkyl group
R³: C₆-C₁₆ (alkyl, alkenyl, alkynyl) or a group selected from

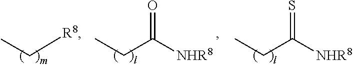

(R⁸=aryl, heteroaryl)
B represents —(CH₂)ⱼCOOR⁹, (CH₂)ⱼCONR⁵OH, —(CH₂)ⱼCONHR⁶, —(CH₂)ⱼX, —(CH₂)ⱼCH═CH₂, (R⁵=phenyl, benzyl, lower alkyl, R⁶=lower alkyl, CH₂CH₂NH₂, R⁹=lower alkyl, X=halogen)
m, l=3-8
n, j=0-3.

### DETAILED DESCRIPTION OF THE INVENTION

Overview of Structures of Compounds of the Invention

Compounds of the invention are constructed based on two zinc-binding functionalities within a distance of 3 to 5 carbon atoms and contain appropriate chemical motifs facilitating interactions with ATX enzyme.

Some of the compounds described herein contain one or more asymmetric centers and may thus give rise to diastereomers and optical isomers. The present invention is meant to include such possible diastereomers as well as their racemic and resolved, enantiomerically pure forms, and pharmaceutically acceptable salts thereof.

The term “alkyl” as used herein refers to the radical of saturated aliphatic groups, and means straight chain alkyl groups, branched chain alkyl groups, cycloalkyl (alicyclic) groups, alkyl substituted cycloalkyl groups, and cycloalkyl substituted alkyl groups. The alkyl groups can be (C₁-C₂₀) alkyl, or (C₁-C₁₀) alkyl or (C₁-C₈) alkyl. Preferred alkyl groups are methyl, ethyl, n-propyl, iso-propyl, n-butyl, sec-butyl, iso-butyl, n-pentyl, n-hexyl, n-heptyl, n-octyl.

The term “lower alkyl” refers to alkyl groups having up to 6 carbons (C₁-C₆).

The term “alkenyl” as used herein refers to the radical of unsaturated alkyl, as defined above, containing at least one double bond.

The term “alkynyl” as used herein refers to the radical of unsaturated alkyl, as defined above, containing at least one triple bond.

The term “lower alkoxy” refers to a lower alkyl group singular bonded to oxygen.

Preferably an aryl group is a C₆₋₁₂ monocyclic or bicyclic hydrocarbon ring wherein at least one ring is aromatic, preferred are phenyl, biphenyl, naphthyl or tetrahydro-naphthalenyl.

The term “heteroaryl” as used herein refers to a 5-6 membered monocyclic aromatic or a fused 8-10 membered bicyclic aromatic ring containing 1 to 4 heteroatoms selected from oxygen, nitrogen and sulphur. Examples of such monocyclic aromatic rings include thienyl, furyl, pyrrolyl, imidazolyl, oxazolyl, thiazolyl, oxadiazolyl, isothiazolyl, isoxazolyl, thiadiazolyl, pyranyl, pyrazolyl, pyrimidyl, pyridazinyl, pyrazinyl and pyridyl.

As mentioned above, the invention also includes pharmaceutically acceptable salts of the above-mentioned compounds (e.g., compounds of formula I). A compound of the invention can possess a sufficiently acidic functionality, a sufficiently basic functionality, or both functional groups. Accordingly, a compound may react with any of a number of inorganic bases, and organic and inorganic acids, to form a pharmaceutically acceptable salt.

The term “pharmaceutically acceptable salt” as used herein refers to salts of the compounds of formula I which are substantially non-toxic to living organisms. Typical pharmaceutically acceptable salts include those salts prepared by reaction of the compounds of the present invention with a pharmaceutically acceptable mineral or organic acid or an inorganic base. Such salts are known as acid addition and base addition salts.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogen phosphate, dihydrogen phosphate, metaphosphate, pyrophosphate, chloride, bromide, iodide, acetate, propionate, decanoate, caprylate, acrylate, formate, isobutyrate, caproate, heptanoate, propionate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylene-sulfonate, phenylacetate, phenyipropionate, phenylbutyrate, citrate, lactate, γ-hydroxybutyrate, glycolate, tartrate, methanesulfonate, propanesulfonate, naphthalene-1-sulfonate, napthalene-2-sulfonate, mandelate and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. Suitable organic bases include trialkylamines such as triethylamine, procaine, dibenzylamine, N-benzyl-β-phenethyl-amine, 1-ephenamine, N,N′-dibenzylethylene-diamine, dehydroabietylamine, N-ethylpiperidine, benzylamine, dicyclohexylamine, or the like pharmaceutically acceptable amines. In an embodiment, the above-mentioned salt is a potassium salt or a sodium salt.

In one aspect, the present invention provide compounds of formula I, wherein A, X, Y, R² and R³ are the same as previously defined.

In another aspect, the present invention provides a composition comprising a compound of formula I and a pharmaceutically acceptable carrier or excipient. The compound of formula I may be administered in the form of pharmaceutical compositions. They can be administered by a variety of routes including oral, rectal, transdermal, subcutaneous, intravenous, intramuscular, intranasal and intrathecal. The compounds are effective as both injectable and oral compositions. Such compositions are prepared in a manner well known in the pharmaceutical art and comprise at least one active compound and a pharmaceutically acceptable diluent or carrier or excipient. Supplementary active compounds can also be incorporated into the compositions.

In making the compositions employed in the present invention, the active ingredient (e.g., a compound of formula I) is usually mixed with a pharmaceutically acceptable carrier or excipient. As used herein “pharmaceutically acceptable carrier” or “excipient” includes any and all solvents, buffers, dispersion media, coatings, antibacterial and antifungal agents, isotonic and absorption delaying agents, and the like that are physiologically compatible. The carrier can be suitable, for example, for intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration (see Remington: The Science and Practice of Pharmacy by Alfonso R. Gennaro, 2003, 21^{th} edition, Mack Publishing Company).

Formulations suitable for oral administration can consist of (a) liquid solutions, such as an effective amount of active agent(s)/composition(s) suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds/compositions of the invention include ethylenevinyl acetate copolymer particles, osmotic pumps, implantable infusion systems, and liposomes. Formulations for inhalation may contain excipients, (e.g., lactose) or may be aqueous solutions containing, for example, polyoxyethylene-9-lauryl ether, glycocholate and deoxycholate, or may be oily solutions for administration in the form of nasal drops, or as a gel.

For preparing pharmaceutical compositions from the compound(s)/composition(s) of the present invention, pharmaceutically acceptable carriers are either solid or liquid. Solid form preparations include powders, tablets, pills, capsules, cachets, suppositories, and dispersible granules. A solid carrier can be one or more substance, which may also act as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired. The powders and tablets may typically contain from 5% or 10% to 70% of the active compound/composition. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use are prepared by dissolving the active compound(s)/composition(s) in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Formulations to be used for in vivo administration are preferably sterile. This is readily accomplished, for example, by filtration through sterile filtration membranes.

The composition may also contain more than one active compound for the particular indication being treated, preferably those with complementary activities that do not adversely affect each other. It may be desirable to use the above-mentioned composition in addition to one or more agents currently used to prevent or treat the disorder in question. The above-mentioned agents may be formulated in a single composition or in several individual compositions which may be co-administered in the course of the treatment.

The amount of the pharmaceutical composition (e.g., a compound of formula I, or a salt thereof) which is effective in the prevention and/or treatment of a particular disease, disorder or condition (e.g., inflammatory disease, neural injury) will depend on the nature and severity of the disease, the chosen prophylactic/therapeutic regimen, the target site of action, the patient's weight, special diets being followed by the patient, concurrent medications being used, the administration route and other factors that will be recognized by those skilled in the art. The dosage will be adapted by the clinician in accordance with conventional factors such as the extent of the disease and different parameters from the patient. Typically, 0.001 to 1000 mg/kg of body weight/day will be administered to the subject. In an embodiment, a daily dose range of about 0.01 mg/kg to about 500 mg/kg, in a further embodiment of about 0.1 mg/kg to about 200 mg/kg, in a further embodiment of about 1 mg/kg to about 100 mg/kg, in a further embodiment of about 10 mg/kg to about 50 mg/kg, may be used. The dose administered to a patient, in the context of the present invention should be sufficient to effect a beneficial prophylactic and/or therapeutic response in the patient over time. The size of the dose also will be determined by the existence, nature, and extent of any adverse side-effects that accompany the administration. Effective doses may be extrapolated from dose response curves derived from in vitro or animal model test systems. For example, in order to obtain an effective mg/kg dose for humans based on data generated from rat studies, the effective mg/kg dosage in rat may be divided by six.

In another aspect, the present invention provides a method for inhibiting ATX activity in a system, e.g., a cell, cell-free system, biological system, or a subject, said method comprising contacting said system with, or administering to said subject, an effective amount of the above-mentioned compound or composition.

In another aspect, the invention provides a method for preventing and/or treating an inflammatory disease or condition in a subject, said method comprising administering to said subject an effective amount of the above-mentioned compound or composition.

In another aspect, the present invention provides the use of the above-mentioned compound or composition for the prevention and/or treatment of an inflammatory disease or condition.

In yet another aspect, the present invention provides the use of the above-mentioned compound or composition for the preparation of a medicament for the prevention and/or treatment of cancer.

In yet another aspect, the present invention provides the use of the above-mentioned compound or composition for the preparation of a medicament for the prevention and/or treatment of obesity or autoimmune diseases.

An “effective amount” refers to an amount effective, at dosages and for periods of time necessary, to achieve the desired prophylactic or therapeutic result. An effective amount refers to the amount of active compound or pharmaceutical agent that elicits the biological or medicinal response in a tissue, system, animal, individual or human that is being sought by a researcher, veterinarian, medical doctor or other clinician, which includes one or more of the following:

(A) Preventing the disease; for example, preventing a neural disease, condition or disorder and/or an inflammatory disease, condition or disorder in an individual that may be predisposed to the disease, condition or disorder but does not yet experience or display the pathology or symptomatology of the disease,

(B) Inhibiting the disease; for example, inhibiting a neural disease, condition or disorder and/or an inflammatory disease, condition or disorder in an individual that is experiencing or displaying the pathology or symptomatology of the disease, condition or disorder (i.e., arresting further development of the pathology and/or symptomatology), and

(C) Ameliorating the disease; for example, ameliorating neural disease, condition or disorder and/or an inflammatory disease, condition or disorder in an individual that is experiencing or displaying the pathology or symptomatology of the disease, condition or disorder (i.e., reversing the pathology and/or symptomatology).

An effective amount of a compound or composition of the present invention may vary according to factors such as the disease state, age, sex, and weight of the individual, and the ability of the compound to elicit a desired response in the individual. Dosage regimens may be adjusted to provide the optimum prophylactic or therapeutic response. An effective amount is also one in which any toxic or detrimental effects of the compound are outweighed by the therapeutically beneficial effects. It will be understood that the amount of the compound actually administered will be determined by a physician, in the light of the relevant circumstances, including the condition to be treated, the chosen route of administration, the actual compound administered, the age, weight, and response of the individual patient, and the severity of the patient's symptoms, and therefore the above dosage ranges are not intended to limit the scope of the invention in any way. In some instances dosage levels below the lower limit of the aforesaid range may be more than adequate, while in other cases still larger doses may be employed without causing any harmful side effect, provided that such larger doses are first divided into several smaller doses for administration throughout the day.

In an embodiment, the above-mentioned treatment may be effected prior to, after, or both prior to and after the onset of symptom(s) of a neural disease or condition. For example, a compound or composition of the invention (e.g., a compound of formula I, or a salt thereof, or a composition comprising a compound of formula I, or a salt thereof and a pharmaceutically-acceptable carrier) may be administered to a subject prior to, after, or both prior to and after the onset of symptom(s) of a neural disease or condition. Similarly, the invention provides a use of a compound or composition of the invention (e.g., a compound of formula I, or a salt thereof, or a composition comprising a compound of formula I, or a salt thereof and a pharmaceutically-acceptable carrier) for the treatment of, or for the preparation of a medicament for the treatment of, a neural disease or condition, wherein the use is prior to, after, or both prior to and after the onset of symptom(s) of the neural disease or condition.

As used herein, the terms “subject” or “patient” are used interchangeably are used to mean any animal, such as a mammal, including humans and non-human primates. In an embodiment, the above-mentioned subject is a mammal. In a further embodiment, the above-mentioned subject is a human.

The articles “a,” “an” and “the” are used herein to refer to one or to more than one (i.e., to at least one) of the grammatical object of the article.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

Definitions provided herein are not intended to be limiting from the meaning commonly understood by one of skill in the art unless indicated otherwise.

In an aspect, the present invention provides a process for preparing a compound of formula I. The key reaction is the following:

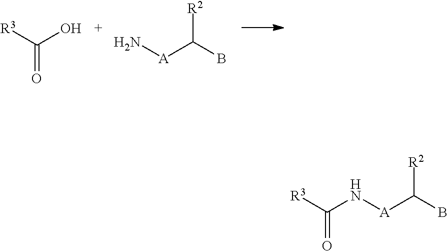

wherein A, B, R² and R³ are the same as previously defined.

In a preferred embodiment, B is —(CH₂)ⱼCOOR⁹ (R⁹=lower alkyl), and the process further comprises a step of converting a compound of formula (IIa) to a hydroxamate of formula (III).

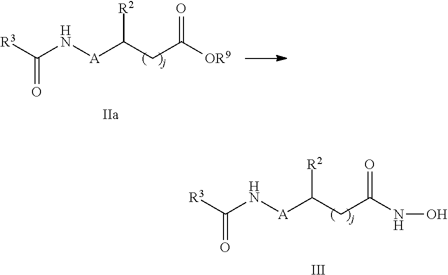

In a preferred embodiment, B is —(CH₂)ⱼCOOR⁹ (R⁹=lower alkyl), and the process further comprising:
(i) hydrolyzing a compound of formula (IIa) to provide the corresponding carboxylic acid of formula (IV), and

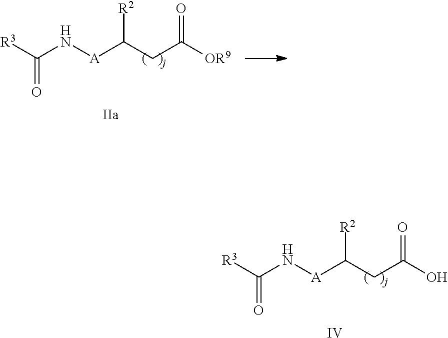

(ii) coupling the compound of formula (IV) with 1,2-phenylenediamine to provide a compound of formula (V).

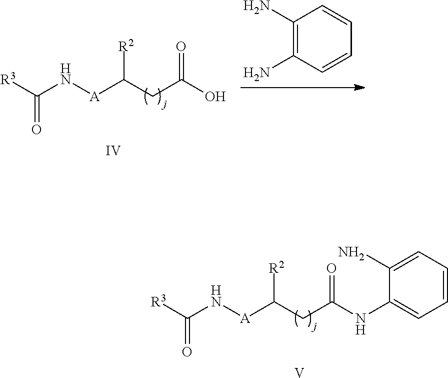

In a preferred embodiment, B is —(CH₂)ⱼX (X=halogen), and the process further comprising:
(1) converting a compound of formula (IIb) into a compound of formula (VI);

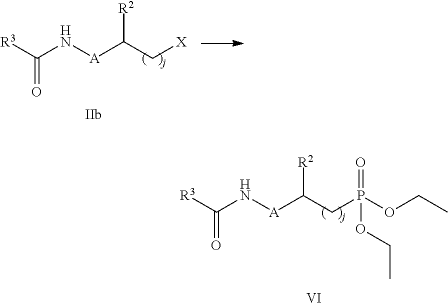

(2) converting a compound of formula (VI) into a compound of formula (VII);

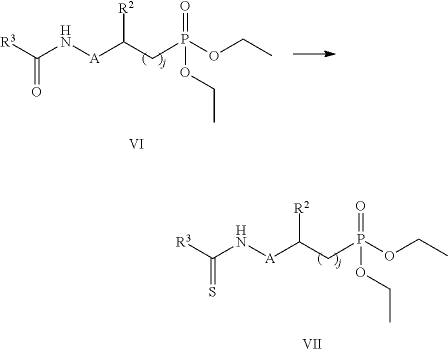

(3) converting a compound of formula (VII) into a compound of formula (VIII).

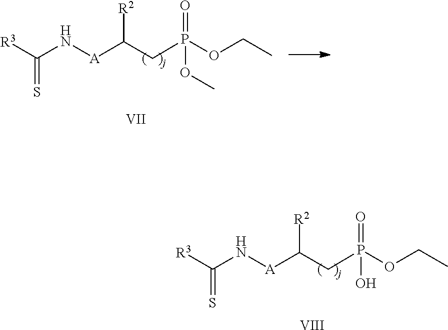

In a preferred embodiment, B is —(CH₂)ⱼCH═CH₂, and the process further comprises steps of:
(a) converting a compound of formula (IIc) into a compound of formula (IX);

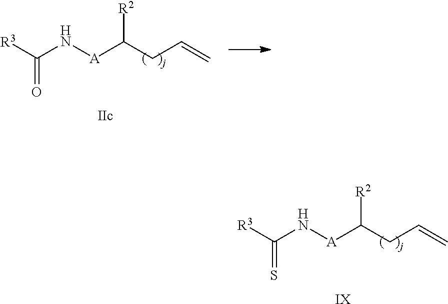

(b) converting a compound of formula (IX) into a compound of formula (X).

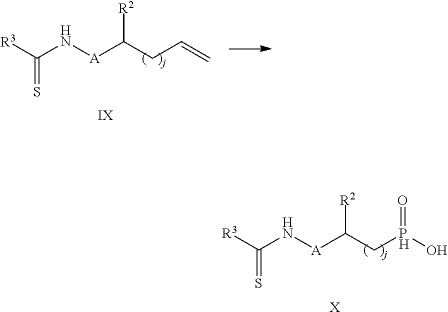

In a preferred embodiment, B is —(CH₂)ⱼCONHR⁶ (R⁶=lower alkyl, CH₂CH₂NH₂, j=0-3), and the process further comprising:
(1) converting a compound of formula (IId) into a compound of formula (XI);

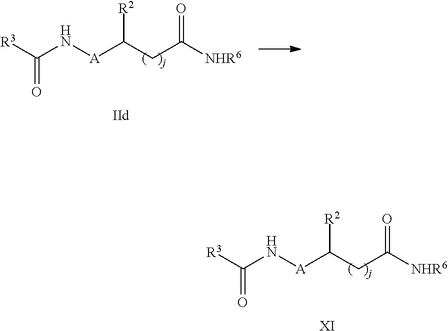

(2) converting a compound of formula (XI) into a compound of formula (XII).

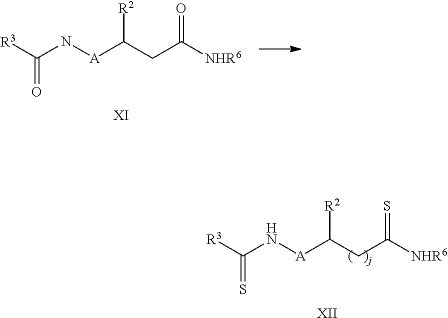

In a preferred embodiment, B is —(CH₂)ⱼCONHR⁹, wherein R⁹ is a C₁-C₈ alkyl group, CH₂CH₂NH₂, j=0-3), and further comprising:
(1) Reacting a compound of formula (IIe) with a compound of formula (IIf) to provide a compound of formula (XII);

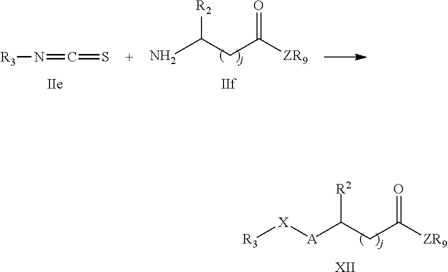

(2) Converting the compound of formula (XII) into a compound of formula (XIII).

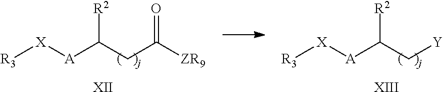

The present invention is illustrated in further details by the following non-limiting examples.

### EXAMPLES

### Example 1

### Synthesis and Characterization of Inhibitors

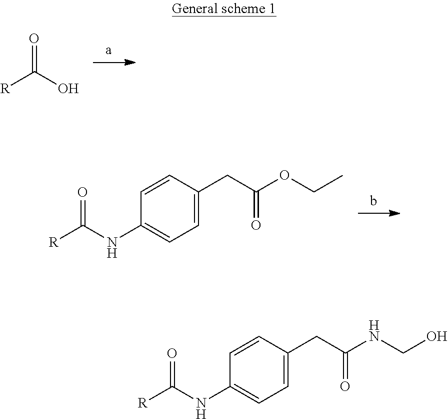

(a) Ethyl-4-aminophenylacetate.HCl, WSCI, Et₃N, DCM, 0° C. to room temperature;

(b) NH₂OH.HCl, EtONa, 0° C. to room temperature.

(1a) Coupling of carboxylic acids with ethyl-4-aminophenylacetate HCl

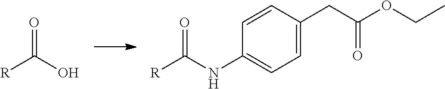

The appropriate carboxylic acid (1.2 mmol), WSCI (1.2 mmol, 230 mg) and Et₃N (2.2 mmol, 0.31 mL) were added to a stirring solution of ethyl-4-aminophenylacetate.HCl (1 mmol) in anhydrous CH₂Cl₂ (10 mL) at 0° C. The solution was stirred for 24 h at room temperature under argon. After washing with H₂O, drying over anhydrous Na₂SO₄ and evaporation of the solvent under reduced pressure, the product was purified by column chromatography.

### Ethyl 2-(4-tetradecanamidophenyl)acetate

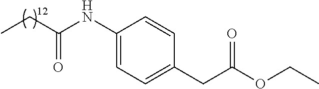

White solid. Yield 48%; mp: 88-90° C.; Eluting mixture used at the column chromatography: petroleum ether/AcOEt 7/3; ¹H NMR (CDCl₃) δ 7.46 (d, J=8.0 Hz, 2H, arom), 7.24 (s, 1H, NH), 7.19 (d, J=8.0 Hz, 2H, arom), 4.13 (q, J=6.0 Hz, 2H, COOCH₂CH₃), 3.56 (s, 2H, PhCH₂CO), 2.33 (t, J=8.0 Hz, 2H, CH₂CO), 1.80-1.50 (m, 2H, CH₂), 1.40-1.10 (m, 23H, CH₂, CH₃), 0.87 (t, J=6.0 Hz, 3H, CH₃); ¹³C NMR (CDCl₃) δ 171.7, 171.4, 138.0, 136.9, 129.8, 119.8, 60.9, 40. 37, 31.9, 29.6, 29.5, 29.4, 29.3, 25.6, 22.7, 14.1; MS (ESI) m/z (%) 390.40 [(M+H)⁺, 100].

### Ethyl 2-(4-(8-oxo-8-(phenylamino)octanamido)phenyl)acetate

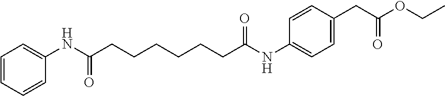 [SMILES: CCOC(=O)Cc1ccc(NC(=O)CCCCCCC(=O)Nc2ccccc2)cc1]

White solid. Yield 71%; mp: 140-142° C.; Eluting mixture used at the column chromatography: petroleum ether/AcOEt 5/5 to 4/6; ¹H NMR (CDCl₃): δ 7.70 (d, J=6.0 Hz, 2H, arom), 7.60-7.40 (m, 3H, arom), 7.40-7.00 (m, 4H, arom), 4.12 (q, J=8.0 Hz, 2H, CH₂CH₃), 3.55 (s, 2H, PhCH₂CO), 2.31 (t, J=8.0 Hz, 4H, 2×CH₂CO), 1.90-1.50 (m, 4H, CH₂), 1.50-1.30 (m, 4H, CH₂), 1.23 (t, J=8.0 Hz, 3H, CH₃); ¹³C NMR (CDCl₃): δ 171.6, 169.6068.1, 136.0, 129.7, 128.9, 124.1, 119.9, 119.8, 60.9, 40.8, 37.3, 28.4, 25.1, 14.2; MS (ESI) m/z (%): 411.31 [(M+H)⁺, 100].

### Ethyl 2-(4-(6-oxo-6-(phenylamino)hexanamido)phenyl)acetate

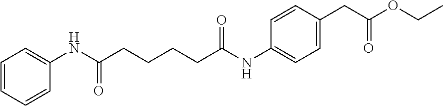 [SMILES: CCOC(=O)Cc1ccc(NC(=O)CCCCC(=O)Nc2ccccc2)cc1]

White solid. Yield 99%, mp: 160-162° C.; Eluting mixture used at the column chromatography: CHCl₃/MeOH 95/5; ¹H NMR (CDCl₃) δ 7.91 (s, 1H, NH), 7.82 (s, 1H, NH), 7.70-7.00 (m, 9H, arom), 4.12 (q, J=6.0 Hz, 2H, CH₂CH₃), 3.55 (s, 2H, PhCH₂CO), 2.60-2.20 (m, 4H, 2×CH₂CO), 1.90-1.50 (m, 4H, CH₂), 1.22 (t, J=6.0 Hz, 3H, CH₃); ¹³C NMR (CDCl₃) δ 172.4, 171.0, 166.4, 138.0, 133.3, 129.6, 128.7, 124.0, 119.9, 119.8, 60.9, 49.7, 48.9, 40.7, 36.6, 24.9, 14.0; MS (ESI) m/z (%) 383.07 [(M+H)⁺, 100].

### ethyl 2-(4-(7-phenylheptanamido)phenyl)acetate

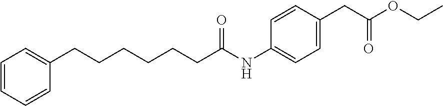 [SMILES: CCOC(=O)Cc1ccc(NC(=O)CCCCCCc2ccccc2)cc1]

Off-white solid. Yield 64%; mp: 80-82° C.; Eluting mixture used at the column chromatography: petroleum ether/AcOEt 6/4; ¹H NMR (CDCl₃) δ 8.30 (s, 1H, NH), 7.48 (d, J=8.0 Hz, 2H, arom), 7.40-7.00 (m, 7H, arom), 4.14 (q, J=6.0 Hz, 2H, OCH₂CH₃), 3.55 (s, 2H, PhCH₂CO), 2.58 (t, J=8.0 Hz, 2H, PhCH₂), 2.30 (t, J=8.0 Hz, 2H, CH₂CONH), 1.90-1.50 (m, 4H, CH₂), 1.50-1.20 (m, 4H, CH₂), 1.24 (t, J=6.0 Hz, 3H, CH₃); ¹³C NMR (CDCl₃) δ 171.8, 171.7, 142.4, 137.1, 129.4, 129.3, 128.1, 128.0, 125.4, 119.9, 60.7, 40.5, 37.2, 35.6, 31.1, 28.9, 28.8, 25.4, 14.0; MS (ESI) m/z (%) 366.26 [(M−H)⁻, 100].

### Ethyl 2-(4-(5-benzamidopentanamido)phenyl)acetate

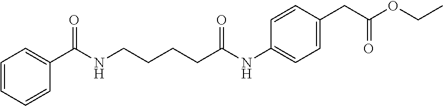 [SMILES: CCOC(=O)Cc1ccc(NC(=O)CCCCNC(=O)c2ccccc2)cc1]

Yellowish solid. Yield 52%; mp: 148-150° C.; Eluting mixture used at the column chromatography: petroleum ether/AcOEt 3/7; ¹H NMR (CDCl₃) δ 8.43 (s, 1H, NH), 7.78 (d, J=8.0 Hz, 2H, arom), 7.60-7.30 (m, 5H, arom), 7.16 (d, J=8.0 Hz, 2H, arom), 6.92 (t, J=6.0 Hz, 1H, NH), 4.11 (q, J=8.0 Hz, 2H, CH₂CH₃), 3.54 (s, 2H, PhCH₂CO), 3.43 (q, J=6.0 Hz, 2H, CH₂NH), 2.37 (t, J=6.0 Hz, 2H, CH₂CO), 1.80-1.40 (m, 4H, CH₂), 1.22 (t, J=8.0 Hz, 3H, CH₂CH₃); ¹³C NMR (CDCl₃) δ 171.8, 171.8, 168.0, 137.3, 134.3, 131.4, 129.6, 129.5, 128.5, 126.9, 119.8, 60.9, 40.7, 39.0, 36.5, 28.8, 22.5, 14.1; MS (ESI) m/z (%) 383.19 [(M+H)⁺, 100].

### Ethyl 2-(4-(5-(4-butoxyphenyl)pentanamido)phenyl)acetate

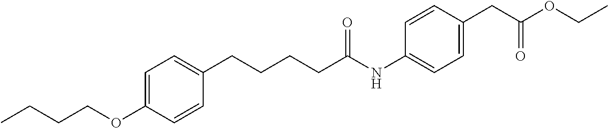 [SMILES: CCCCOc1ccc(CCCCC(=O)Nc2ccc(CC(=O)OCC)cc2)cc1]

Off-white solid. Yield 49%; mp: 132-134° C.; Eluting mixture used at the column chromatography: petroleum ether/AcOEt 8/2; ¹H NMR (CDCl₃) δ 7.43 (d, J=8.0 Hz, 2H, arom), 7.40-7.10 (m, 2H, arom), 7.06 (d, J=8.0 Hz, 2H, arom), 6.80 (d, J=8.0 Hz, 2H, arom), 6.69 (d, J=8.0 Hz, 2H, NH), 4.12 (q, J=8.0 Hz, 2H, OCH₂CH₃), 3.91 (t, J=6.0 Hz, 2H, OCH₂CH₂), 3.55 (s, 2H, PhCH₂CO), 2.57 (t, J=8.0 Hz, 2H, PhCH₂), 2.33 (t, J=8.0 Hz, 2H, CH₂CONH), 1.90-1.30 (m, 8H, CH₂), 1.23 (t, J=8.0 Hz, 3H, OCH₂CH₃), 0.95 (t, J=8.0 Hz, 3H, CH₃); ¹³C NMR (CDCl₃) δ 171.6, 171.1, 157.3, 136.8, 133.9, 129.8, 129.2, 119.8, 114.3, 67.6, 60.9, 40.8, 37.6, 34.7, 31.2, 29.7, 25.2, 19.3, 14.2, 13.9; MS (ESI) m/z (%) 429.25 [(M+NH₄)⁺, 90].

### Ethyl 2-(4-(2-(4-octylphenoxy)acetamido)phenyl)acetate

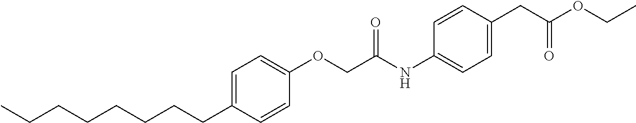 [SMILES: CCCCCCCCc1ccc(OCC(=O)Nc2ccc(CC(=O)OCC)cc2)cc1]

Off-white solid. Yield 40%; mp: 80-82° C.; Eluting mixture used at the column chromatography: petroleum ether/AcOEt 5/5; ¹H NMR (CDCl₃) δ 8.27 (s, 1H, NHCO), 7.53 (d, J=8.0 Hz, 2H, arom), 7.36 (d, J=8.0 Hz, 2H, arom), 7.13 (d, J=8.0 Hz, 2H, arom), 6.88 (d, J=8.0 Hz, 2H, arom), 4.56 (s, 2H, OCH₂CO), 4.13 (q, J=8.0 Hz, 2H, OCH₂CH₃), 3.57 (s, 2H, PhCH₂CO), 2.55 (t, J=6.0 Hz, 2H, CH₂Ph), 1.70-1.50 (m, 2H, CH₂), 1.50-1.10 (m, 10H, CH₂), 1.23 (t, J=8.0 Hz, 3H, OCH₂CH₃), 0.86 (t, J=8.0 Hz, 3H, CH₃); ¹³C NMR (CDCl₃) δ 171.5, 166.4, 155.0, 137.0, 135.8, 130.6, 129.9, 129.6, 120.2, 114.6, 67.8, 60.9, 40.8, 35.0, 31.8, 31.6, 29.4, 29.2, 22.6, 14.2, 14.1; MS (ESI) m/z (%) 424.35 [(M−H)⁻, 100].

### Ethyl 2-(4-(3-(4-octylphenyl)propanamido)phenyl)acetate

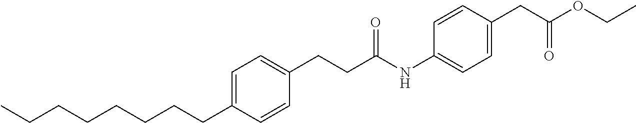 [SMILES: CCCCCCCCc1ccc(CCC(=O)Nc2ccc(CC(=O)OCC)cc2)cc1]

White solid. Yield 56%; mp: 99-101° C.; Eluting mixture used at the column chromatography: petroleum ether/AcOEt 7/3 to 6/4; ¹H NMR (CDCl₃) δ 8.00 (s, 1H, NH), 7.38 (d, J=8.0 Hz, 2H, arom), 7.20-7.00 (m, 6H, arom), 6.92 (t, J=6.0 Hz, 1H, NH), 4.12 (q, J=8.0 Hz, 2H, OCH₂CH₃), 3.54 (s, 2H, PhCH₂CO), 2.97 (t, J=8.0 Hz, 2H, CH₂Ph), 2.58 (q, J=8.0 Hz, 4H, PhCH₂CH₂CO), 1.70-1.40 (m, 2H, CH₂), 1.40-1.20 (m, 10H, CH₂), 1.23 (t, J=8.0 Hz, 3H, OCH₂CH₃), 0.89 (t, J=8.0 Hz, 3H, CH₃); ¹³C NMR (CDCl₃) S 171.8, 170.8, 140.7, 137.7, 136.9, 129.5, 128.4, 128.1, 120.1, 60.8, 40.6, 39.1, 35.4, 31.8, 31.5, 31.0, 29.4, 29.3, 29.1, 22.5, 14.0; MS (ESI) m/z (%) 422.36 [(M−H)⁻, 100].

### Ethyl 2-(4-(5-(4-methoxybenzamido)pentanamido)phenyl)acetate

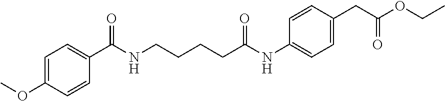 [SMILES: CCOC(=O)Cc1ccc(NC(=O)CCCCNC(=O)c2ccc(OC)cc2)cc1]

White solid. Yield 39%; mp: 167-169° C.; Eluting mixture used at the column chromatography: CHCl₃/MeOH 97/3 to 95/5; ¹H NMR (CDCl₃): δ 8.62 (s, 1H, PhNHCO), 7.90-7.60 (m, 2H, arom), 7.60-7.40 (m, 2H, arom), 7.20-7.00 (m, 2H, arom), 6.95-6.90 (m, 1H, CONHCH₂), 6.90-6.70 (m, 2H, arom), 4.10 (q, J=8.0 Hz, 2H, OCH₂CH₃), 3.79 (s, 3H, OCH₃), 3.53 (s, 2H, PhCH₂CO), 3.50-3.20 (m, 2H, NHCH₂CH₂), 2.50-2.20 (m, 2H, CH₂CH₂CO), 1.80-1.30 (m, 4H, CH₂), 1.21 (t, J=8.0 Hz, 3H, OCH₂CH₃); ¹³C NMR (CDCl₃): δ 171.9, 167.5, 162.1, 155.2 137.4, 129.6, 129.4, 128.8, 126.6, 119.9, 113.6, 60.9, 55.3, 40.7, 39.0, 36.5, 28.9, 22.6, 14.1; MS (ESI) m/z (%): 413.12 [(M+H)⁺, 100].

(1b) Synthesis of Hydroxamic Acids

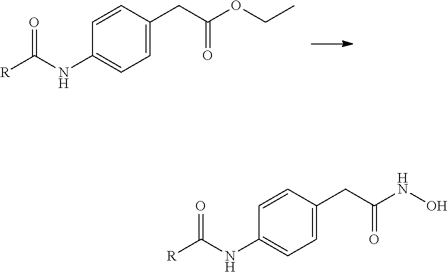

To a stirred solution of the ester (1 mmol) in EtOH (4 mL), which was cooled at 0° C., NH₂OH.HCl (10 mmol, 695 mg) and a solution of 21% w/w EtONa (20 mmol, 1.36 g) in EtOH (7.25 mL) were added. The solution was stirred at room temperature for 2-24 h under argon and quenched by adding HCl 6N. The pH was adjusted at 8 by addition of 1N NaOH, and the solid was collected after filtration and washings with CHCl₃, MeOH and Et₂O and dried.

### N-(4-(2-(hydroxyamino)-2-oxoethyl)phenyl)tetradecanamide

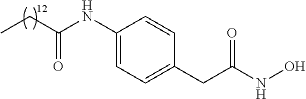

Off-white solid. Yield 45%; mp: 112-114° C.; ¹H NMR (DMSO) δ 10.74 (s, 1H, NHOH), 9.90 (s, 1H, NH), 8.94 (s, 1H, NHOH), 7.47 (d, J=8.0 Hz, 2H, arom), 7.14 (d, J=8.0 Hz, 2H, arom), 3.20 (s, 2H, PhCH₂CO), 2.25 (t, J=8.0 Hz, 2H, CH₂CO), 1.70-1.40 (m, 2H, CH₂), 1.40-1.00 (m, 20H, CH₂,), 0.82 (t, J=6.0 Hz, 3H, CH₃); ¹³C NMR (CD₃OD) δ 169.3, 159.6, 132.4, 130.7, 129.9, 121.2, 41.4, 40.2, 37.8, 34.8, 33.7, 33.1, 30.8, 30.7, 30.5, 27.0, 24.1, 23.8, 22.7, 14.5; MS (ESI) m/z (%) 375.40 [(M−H)⁻, 100]; HRMS 375.2675 (M−H)⁻, calcd. 375.2653.

### N¹-(4-(2-(hydroxyamino)-2-oxoethyl)phenyl)-N⁶-phenyladipamide

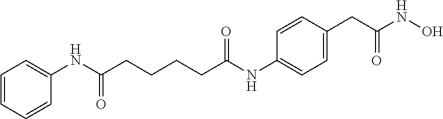 [SMILES: O=C(Cc1ccc(NC(=O)CCCCC(=O)Nc2ccccc2)cc1)NO]

Off-white solid. Yield 50%; mp: 253-255° C.; ¹H NMR (DMSO) δ 10.20 (s, 1H, NH), 10.16 (s, 1H, NH), 8.49 (s, 1H, NHOH), 7.80-7.40 (m, 4H, arom), 7.40-7.10 (m, 4H, arom), 7.10-6.90 (m, 1H, arom), 3.23 (s, 2H, PhCH₂CO), 2.40-2.20 (m, 4H, 2×CH₂CO), 1.80-1.30 (m, 4H, CH₂); ¹³C NMR (DMSO) δ 171.4, 171.2, 167.1, 139.5, 137.9, 130.7, 129.2, 128.7, 123.0, 119.1, 119.0, 36.2, 30.8, 24.9; MS (ESI) m/z (%) 368.40 [M−H)⁻, 100]; HRMS 368.1615 (M−H)⁻, calcd. 368.1616.

### N-(4-(2-(hydroxyamino)-2-oxoethyl)phenyl)-7-phenylheptanamide

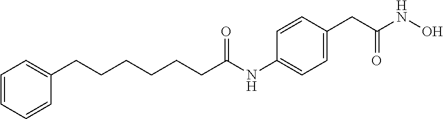 [SMILES: O=C(Cc1ccc(NC(=O)CCCCCCc2ccccc2)cc1)NO]

Off-white solid. Yield 47%; mp: 176-178° C.; ¹H NMR (DMSO) δ 10.64 (s, 1H, NHOH), 9.85 (s, 1H, NHCO), 8.84 (s, 1H, NHOH), 7.51 (d, J=8.0 Hz, 2H, arom), 7.40-6.90 (m, 7H, arom), 3.22 (s, 2H, PhCH₂CO), 2.56 (t, J=8.0 Hz, 2H, PhCH₂), 2.27 (t, J=8.0 Hz, 2H, CH₂CONH), 1.70-1.50 (m, 4H, CH₂), 1.40-1.00 (m, 4H, CH₂); ¹³C NMR (DMSO) δ 171.2, 167.1, 142.3, 137.9, 130.6, 129.2, 128.3, 125.7, 119.0, 36.4, 35.2, 31.0, 28.5, 25.2; MS (ESI) m/z (%) 353.24 [(M−H)⁻, 100]; HRMS 353.1872 (M−H)⁻, calcd. 353.1871.

### N-(5-((4-(2-(hydroxyamino)-2-oxoethyl)phenypamino)-5-oxopentyl)benzamide

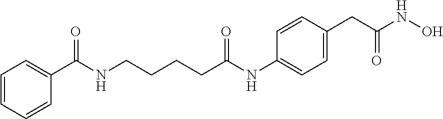 [SMILES: O=C(Cc1ccc(NC(=O)CCCCNC(=O)c2ccccc2)cc1)NO]

Off-white solid. Yield 39%, mp: 179-181° C.; ^{t}H NMR (DMSO) δ 10.68 (s, 1H, NHOH), 9.90 (s, 1H, NHCO), 8.87 (s, 1H, NHOH), 8.51 (t, J=6.0 Hz, 1H, NHCH₂), 7.82 (d, J=8.0 Hz, 2H, arom), 7.60-7.30 (m, 5H, arom), 7.14 (d, J=8.0 Hz, 2H, arom), 3.27 (q, J=6.0 Hz, 2H, CH₂NH), 3.20 (s, 2H, PhCH₂CO), 2.31 (t, J=6.0 Hz, 2H, CH₂CO), 1.80-1.40 (m, 4H, CH₂); ¹³C NMR (DMSO) δ 171.4, 167.5, 166.4, 137.9, 134.8, 131.3, 130.7, 129.3, 128.5, 127.3, 119.2, 40.2, 39.8, 36.7, 29.4, 23.4; MS (ESI) m/z (%) 368.10 [(M−H)⁻, 100]; HRMS 368.1615 (M−H)⁻, calcd. 368.1616.

### 5-(4-butoxyphenyl)-N-(4-(2-(hydroxyamino)-2-oxoethyl)phenyl)pentanamide

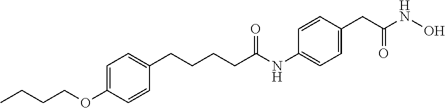 [SMILES: CCCCOc1ccc(CCCCC(=O)Nc2ccc(CC(=O)NO)cc2)cc1]

Off-white solid. Yield 16%; mp: 198-200° C.; ¹H NMR (DMSO): δ 10.62 (s, 1H, NHOH), 9.83 (s, 1H, NHCO), 8.81 (s, 1H, NHOH), 7.48 (d, J=8.0 Hz, 2H, arom), 7.30-6.90 (m, 4H, arom), 6.81 (d, J=6.0 Hz, 2H, arom), 3.90 (t, J=6.0 Hz, 2H, OCH₂), 3.20 (s, 2H, PhCH₂CO), 2.29 (t, J=8.0 Hz, 2H, CH₂CONH), 1.80-1.20 (m, 10H, CH₂), 0.91 (t, J=8.0 Hz, 3H, CH₃); ¹³C NMR (DMSO): δ 171.1, 167.2, 156.8, 137.8, 133.8, 130.5, 129.1, 119.0, 114.2, 67.0, 36.3, 34.1, 30.9, 24.8, 18.8, 13.7; MS (ESI) m/z (%): 397.34 [(M−H)⁻, 100]; HRMS 397.2126 (M−H)⁻, calcd. 397.2133.

### N-hydroxy-2-(4-(2-(4-octylphenoxy)acetamido)phenyl)acetamide

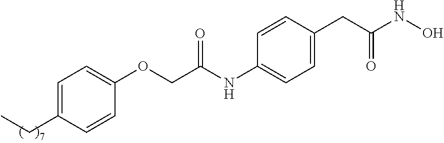

White solid. Yield 29%; mp: 182-184° C.; ¹H NMR (DMSO) δ 10.68 (s, 1H, NHOH), 9.97 (s, 1H, NHCO), 8.81 (s, 1H, NHOH), 7.70-7.40 (m, 2H, arom), 7.40-7.00 (m, 4H, arom), 7.00-6.80 (m, 2H, arom), 4.63 (s, 2H, OCH₂CO), 3.25 (s, 2H, PhCH₂CO), 2.50 (t, J=6.0 Hz, 2H, CH₂Ph), 1.70-1.40 (m, 2H, CH₂), 1.40-1.00 (m, 10H, CH₂), 0.85 (t, J=6.0 Hz, 3H, CH₃); ¹³C NMR (DMSO) δ 167.4, 166.8, 156.0, 136.9, 135.2, 131.5, 129.3, 119.8, 114.6, 67.4, 34.4, 31.4, 29.0, 28.8, 22.3, 14.0; MS (ESI) m/z (%) 411.16 [(M−H)⁻, 100]; HRMS 411.2289 (M−H)⁻, calcd. 411.2289.

### N-(4-(2-(hydroxyamino)-2-oxoethyl)phenyl)-3-(4-octylphenyl)propanamide

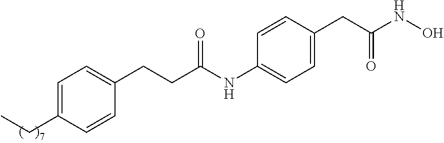

Yellowish solid. Yield 58%; mp: 195-197° C.; ¹H NMR (DMSO) δ 10.67 (s, 1H, NHOH), 9.90 (s, 1H, NHCO), 8.88 (s, 1H, NHOH), 7.48 (d, J=8.0 Hz, 2H, arom), 7.30-6.90 (m, 6H, arom), 3.21 (s, 2H, PhCH₂CO), 2.85 (t, J=6.0 Hz, 2H, CH₂Ph), 2.57 (t, J=6.0 Hz, 4H, PhCH₂CH₂CO), 1.70-1.40 (m, 2H, CH₂), 1.40-1.00 (m, 10H, CH₂), 0.84 (t, J=6.0 Hz, 3H, CH₃); ¹³C NMR (DMSO) δ 170.6, 167.5, 140.1, 138.5, 137.8, 130.8, 129.3, 128.4, 128.3, 119.2, 35.0, 31.5, 31.2, 30.7, 29.0, 28.9, 22.3, 14.2; MS (ESI) m/z (%) 409.19 [(M−H)⁻, 100]; HRMS 409.2497 (M−H)⁻, calcd. 408.2497.

### N¹-(4-(2-(hydroxyamino)-2-oxoethyl)phenyl)-N⁸-phenyloctanediamide

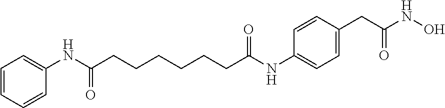 [SMILES: O=C(Cc1ccc(NC(=O)CCCCCCC(=O)Nc2ccccc2)cc1)NO]

Yellowish solid. Yield 96%; mp: 195-197° C.; ¹H NMR (DMSO) δ 10.64 (s, 1H, NHOH), 9.84 (s, 2H, 2×NH), 8.83 (s, 1H, NHOH), 7.70-7.40 (m, 4H, arom), 7.40-7.10 (m, 4H, arom), 7.10-6.90 (m, 1H, arom), 3.22 (s, 2H, PhCH₂CO), 2.40-2.10 (m, 4H, 2×CH₂CO), 1.80-1.40 (m, 4H, CH₂), 1.40-1.10 (m, 4H, CH₂); ¹³C NMR (DMSO) δ 171.3, 171.1, 167.2, 139.4, 137.8, 130.5, 129.1, 128.7, 123.0, 119.1, 119.0, 38.8, 36.4, 28.6, 25.1; MS (ESI) m/z (%) 396.25 [(M−H)⁻, 100]; HRMS 396.1932 (M−H)⁻, calcd. 396.1929.

### N-(5-((4-(2-(hydroxyamino)-2-oxoethyl)phenyl)amino)-5-oxopentyl)-4-methoxy benzamide

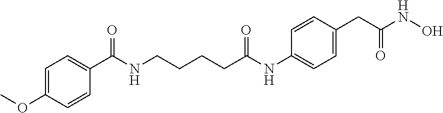 [SMILES: COc1ccc(C(=O)NCCCCC(=O)Nc2ccc(CC(=O)NO)cc2)cc1]

Yellowish solid. Yield 100%; mp: 216-218° C.; ¹H NMR (DMSO) δ 9.85 (s, 1H, NHCO), 8.34 (s, 1H, NHOH), 7.81 (d, J=8.0 Hz, 2H, arom), 7.50 (d, J=8.0 Hz, 2H, arom), 7.14 (d, J=8.0 Hz, 2H, arom), 6.97 (d, J=8.0 Hz, 2H, arom), 3.79 (s, 3H, OCH₃), 3.50-3.30 (m, 2H, NHCH₂CH₂), 3.20 (s, 2H, PhCH₂CO), 2.40-2.10 (m, 2H, CH₂CH₂CO), 1.80-1.30 (m, 4H, CH₂); ¹³C NMR (DMSO) δ 171.1, 167.2, 165.6, 161.5, 137.8, 55.4, 38.9, 38.3, 36.1, 29.0, 22.8; MS (ESI) m/z (%) 398.18 [(M−H)⁻, 100]; HRMS 398.1724 (M−H)⁻, calcd. 398.1721.

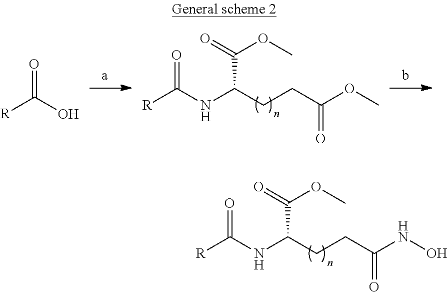

(a) Diethyl-L-glutamate.HCl, WSCI, Et₃N, DCM, 0° C. to room temperature; (b) NH₂OH.HCl, EtONa, EtOH, 0° C. to room temperature.

(2a) Coupling of Carboxylic Acids with dimethyl-L-glutamate

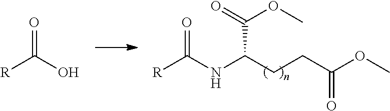

The appropriate carboxylic acid (1.2 mmol), WSCI (1.2 mmol, 230 mg) and Et₃N (2.2 mmol, 0.31 mL) were added to a stirring solution of dimethyl-L-glutamate.HCl (1 mmol) in anhydrous CH₂Cl₂ (10 mL) at 0° C. The solution was stirred for 24 h at room temperature under argon. After washing with H₂O, drying over anhydrous Na₂SO₄ and evaporation of the solvent under reduced pressure, the product was purified by column chromatography.

### Dimethyl (6-oxo-6-(phenylamino)hexanoyl)-L-glutamate

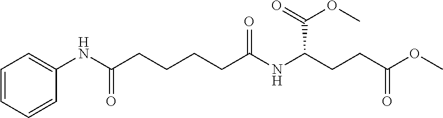 [SMILES: COC(=O)CC[C@H](NC(=O)CCCCC(=O)Nc1ccccc1)C(=O)OC]

White solid. Yield 99%; mp: 116-118° C.; Eluting mixture used at the column chromatography: CHCl₃/MeOH 95/5 to 9/1; ¹H NMR (CDCl₃) δ 8.41 (s, 1H, NH), 7.53 (d, J=6.0 Hz, 2H, arom), 7.26 (t, J=6.0 Hz, 2H, arom), 7.04 (t, J=6.0 Hz, 1H, arom), 6.83 (d, J=8.0 Hz, 2H, NH), 4.56 (t, J=8.0 Hz, 1H, CH), 3.69 (s, 3H, OCH₃), 3.63 (s, 3H, OCH₃), 2.80-2.00 (m, 8H, CH₂), 1.80-1.50 (m, 4H, CH₂); ¹³C NMR (CDCl₃) δ 173.3, 173.2, 172.4, 171.5, 138.2, 128.8, 123.9, 119.7, 52.5, 51.9, 51.6, 36.8, 35.5, 30.1, 26.7, 24.8, 24.5; MS (ESI) m/z (%) 379.21 [(M+H)⁺, 100]; [α]_{D}²⁵=+11.1 (c=1, CHCl₃).

### Dimethyl (6-((4-methoxyphenyl)amino)-6-oxohexanoyl)-L-glutamate

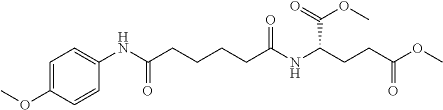

Solid. Yield 70%; mp: 127-129° C.; Eluting mixture used at the column chromatography: CHCl₃/MeOH 98/2 to 95/5; ¹H NMR (CDCl₃) δ 8.74 (s, 1H, NH), 7.30 (d, J=8.0 Hz, 2H, arom), 7.22 (d, J=8.0 Hz, 1H, NH), 6.65 (d, J=8.0 Hz, 2H, arom), 4.50-4.30 (m, 1H, CH), 3.60 (s, 3H, OCH₃), 3.55 (s, 3H, OCH₃), 3.50 (s, 3H, OCH₃), 2.50-1.80 (m, 8H, CH₂), 1.70-1.40 (m, 4H, CH₂); ¹³C NMR (CDCl₃) δ 173.2, 172.9, 172.1, 171.3, 155.6, 131.2, 121.5, 113.5, 5.0, 52.0, 51.4, 51.3, 36.2, 35.2, 29.8, 26.3, 24.7, 24.6; MS (ESI) m/z (%) 431.21 [(M+Na)⁺, 100]; [α]_{D}²⁵=+5.3 (c=0.83, CHCl₃).

### Dimethyl (S)-2-(6-oxo-6-(phenylamino)hexanamido)hexanedioate

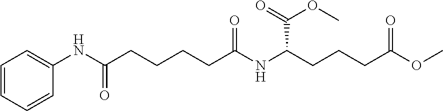 [SMILES: COC(=O)CCC[C@H](NC(=O)CCCCC(=O)Nc1ccccc1)C(=O)OC]

Yellowish oil. Yield 67%; Eluting mixture used at the column chromatography: DCM/MeOH 95/5 to 9/1; ¹H NMR (CDCl₃) δ 8.47 (s, 1H, NH), 7.53 (d, J=8.0 Hz, 2H, arom), 7.24 (t, J=8.0 Hz, 2H, arom), 7.01 (t, J=8.0 Hz, 1H, arom), 6.92 (d, J=8.0 Hz, 1H, NH), 4.70-4.40 (m, 1H, CH), 3.68 (s, 3H, OCH₃), 3.60 (s, 3H, OCH₃), 2.80-2.00 (m, 8H, CH₂), 2.00-1.50 (m, 6H, CH₂); ¹³C NMR (CDCl₃) δ 173.3, 173.2, 172.5, 171.6, 138.4, 128.7, 123.7, 119.7, 52.6, 51.8, 51.6, 36.7, 35.4, 30.9, 30.1, 26.8, 25.2, 24.8; MS (ESI) m/z (%) 393.14 [(M+H)⁺, 98]; [α]_{D}²⁵=−1.2 (c=0.83, CHCl₃).

(2b) Synthesis of Hydroxamic Acids

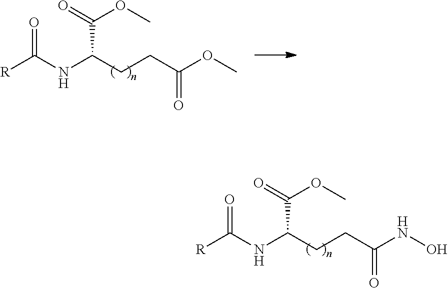

To a stirred solution of the ester (1 mmol) in absolute MeOH (4 mL), which was cooled at 0° C., NH₂OH.HCl (10 mmol, 695 mg) and a solution of 25% w/v MeONa (20 mmol, 1.08 g) in MeOH (4.2 mL) were added. The solution was stirred at room temperature for 48-72 hours under argon and quenched by adding HCl 6N. The pH was adjusted at 8 by the addition of 1N NaOH and the organic solvent was evaporated in vacuo. The residue was extracted with CHCl₃ and the organic layer was dried over anhydrous Na₂SO₄, the solvent was evaporated and the crude product was purified using flash column chromatography eluting with CHCl₃:MeOH 9:1.

### Methyl N⁵-hydroxy-N²-(6-oxo-6-(phenylamino)hexanoyl)-L-glutaminate

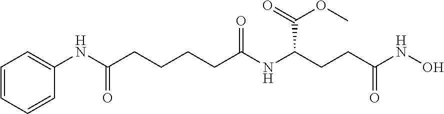 [SMILES: COC(=O)[C@H](CCC(=O)NO)NC(=O)CCCCC(=O)Nc1ccccc1]

Off-white solid. Yield 10%; mp: 118-120° C.; ¹H NMR (CD₃OD) δ 7.80 (s, 1H, NH), 7.43 (d, J=8.0 Hz, 2H, arom), 7.17 (t, J=8.0 Hz, 2H, arom), 6.97 (t, J=8.0 Hz, 1H, arom), 4.30-4.00 (m, 1H, CH), 3.52 (s, 3H, OCH₃), 2.50-1.70 (m, 8H, CH₂), 1.70-1.40 (m, 4H, CH₂); ¹³C NMR (CD₃OD) δ 175.8, 174.7, 174.2, 170.6, 139.9, 129.8, 125.1, 121.1, 52.2, 51.5, 37.6, 36.4, 30.9, 28.3, 27.3, 26.4; MS (ESI) m/z (%) 378.21 [(M−H)⁻, 100]; HRMS 378.1677 (M−H)⁻, calcd. 378.1671; [α]_{D}²⁵=−11.6 (c=0.43, MeOH).

### Methyl N⁵-hydroxy-N²-(6-((4-methoxyphenyl)amino)-6-oxohexanoyl)-L-glutaminate

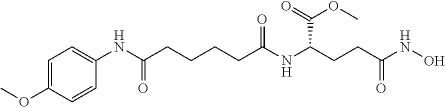 [SMILES: COC(=O)[C@H](CCC(=O)NO)NC(=O)CCCCC(=O)Nc1ccc(OC)cc1]

Off-white solid. Yield 6%; mp: 157-159° C.; ¹H NMR (CD₃OD) δ 7.86 (s, 1H, NH), 7.40 (d, J=8.0 Hz, 2H, arom), 6.82 (d, J=8.0 Hz, 2H, arom), 4.30-4.10 (m, 1H, CH), 3.72 (s, 3H, OCH₃), 3.61 (s, 3H, OCH₃), 2.50-1.80 (m, 8H, CH₂), 1.80-1.50 (m, 4H, CH₂); ¹³C NMR (CD₃OD) δ 175.8, 174.7, 174.0, 170.6, 157.8, 132.8, 123.1, 114.9, 55.8, 52.2, 51.5, 37.4, 36.4, 31.0, 28.3, 26.5, 26.4; MS (ESI) m/z (%) 409.16 [(M−H)⁻, 100]; HRMS 408.1789 (M−H)⁻, calcd. 408.1776; [α]_{D}²⁵=−17.6 (c=0.5, MeOH).

### Methyl (S)-6-(hydroxyamino)-6-oxo-2-(6-oxo-6-(phenylamino) hexanamido) hexanoate

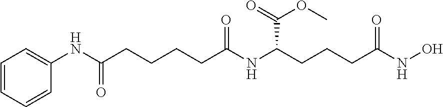 [SMILES: COC(=O)[C@H](CCCC(=O)NO)NC(=O)CCCCC(=O)Nc1ccccc1]

Off-white solid. Yield 10%; mp: 118-120° C.; ¹H NMR (CD₃OD) S 7.53 (d, J=8.0 Hz, 2H, arom), 7.27 (t, J=8.0 Hz, 2H, arom), 7.06 (t, J=8.0 Hz, 1H, arom), 4.40-4.10 (m, 1H, CH), 3.62 (s, 3H, OCH₃), 2.60-2.20 (m, 8H, CH₂), 1.90-1.50 (m, 5H, CH₂); ¹³C NMR (CD₃OD) δ 175.8, 175.4, 174.2, 170.9, 139.8, 129.7, 125.1, 121.2, 53.3, 52.0, 37.5, 36.4, 34.0, 32.4, 31.7, 26.4, 22.2; MS (ESI) m/z (%) 392.27 [(M−H)⁻, 100]; HRMS 392.1814 (M−H)⁻, calcd. 392.1827; [α]_{D}²⁵=−15.6 (c=0.4, MeOH).

### Methyl (S)-5-(6-oxo-6-(phenylamino)hexanamido)nonanoate

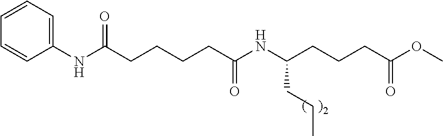

White solid. Yield 66%; m.p. 120-122° C.; ¹H NMR (CDCl₃) δ 9.20 (s, 1H, NH), 7.50 (d, J=8.0 Hz, 2H, arom), 7.15 (t, J=8.0 Hz, 2H, arom), 6.94 (t, J=8.0 Hz, 1H, arom), 6.40 (d, J=8.0 Hz, 1H, NH), 3.90-3.60 (m, 1H, CH), 3.53 (s, 3H, OCH₃), 2.50-2.00 (m, 8H, CH₂), 1.90-1.40 (m, 6H, CH₂), 1.40-1.00 (m, 6H, CH₂), 0.80 (t, J=8.0 Hz, 3H, CH₃); ¹³C NMR (CDCl₃) δ 174.2, 173.1, 172.1, 138.5, 128.4, 123.4, 119.7, 52.8, 51.2, 36.2, 34.1, 33.4, 31.1, 27.9, 27.1, 25.0, 22.3, 21.1, 19.4, 13.8; MS (ESI) m/z (%) 389.2 [(M−H)⁻, 100]; [α]_{D}²⁵=+1.5 (c=1, CHCl₃).

### (S)-N¹-(1-(Hydroxyamino)-1-oxononan-5-yl)-N⁶-phenyladipamide

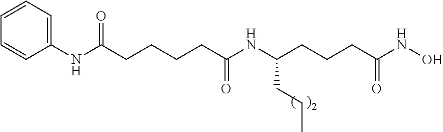

Yellowish solid. Yield 47%; m.p. 161-163° C.; NMR (CD₃OD) δ 7.78 (d, J=10.0 Hz, 1H, NH), 7.54 (d, J=6.0 Hz, 2H, arom), 7.28 (t, J=6.0 Hz, 2H, arom), 7.06 (t, J=6.0 Hz, 1H, arom), 3.80-3.60 (m, 1H, CH), 2.60-2.00 (m, 6H, CH₂), 1.85 (t, J=6.0 Hz, 2H, CH₂), 1.80-1.60 (m, 4H, CH₂), 1.60-1.10 (m, 8H, CH₂), 0.86 (t, J=4.0 Hz, 3H, CH₃); ¹³C NMR (CD₃OD) δ 175.6, 174.1, 172.6, 139.8, 129.7, 125.0, 121.1, 52.0, 37.6, 36.9, 35.7, 35.3, 33.3, 29.3, 26.8, 26.5, 23.5, 23.2, 14.4; MS (ESI) m/z (%) 390.2 [(M−H)⁻, 100]; HRMS 390.2392 (M−H)⁻, (390.2398); [α]_{D}²⁵=−1.1 (c=1, MeOH).

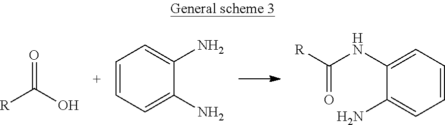

### WSCI, Et₃N, THF, 0° C. to room temperature.

(3a) Coupling of 1,2-phenylenediamine with Acid

1,2-Phenylenediamine (6 mmol, 649 mg) and WSCI (0.75 mmol, 144 mg) were added to a solution of the carboxylic acid (1 mmol) in anhydrous THF (10 mL) and the mixture was stirred under argon atmosphere for 24h. The solvent was evaporated under reduced pressure and AcOEt was added, which was then washed with H₂O and brine and dried over anhydrous Na₂SO₄. The solvent was evaporated and the product was purified by column chromatography (eluting system CHCl₃/MeOH 95/5).

### N¹-(4-(2-((2-aminophenyl)amino)-2-oxoethyl)phenyl)-N⁶-phenyladipamide

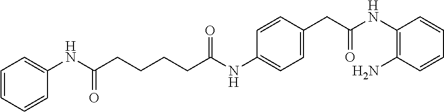 [SMILES: Nc1ccccc1NC(=O)Cc1ccc(NC(=O)CCCCC(=O)Nc2ccccc2)cc1]

Yellowish solid. Yield 20%; mp: 238-240° C.; ¹H NMR (CD₃OD) δ 7.88 (s, 1H, CONH), 7.47 (d, J=8.0 Hz, 4H, arom), 7.40-7.10 (m, 5H, arom), 7.10-6.80 (m, 4H, arom), 3.67 (s, 2H, NH₂), 3.61 (s, 2H, PhCH₂CO), 2.50-2.10 (m, 4H, 2×CH₂CO), 1.90-1.50 (m, 4H, CH₂); ¹³C NMR (CD₃OD) δ 174.2, 171.5, 168.9, 138.8, 130.5, 129.8, 129.5, 125.1 123.2, 121.4, 121.2, 118.5, 116.7, 112.5, 41.1, 37.7, 30.9, 26.5, 23.7; MS (ESI) m/z (%) 445.24 [(M+H)⁺, 100]; HRMS 443.2083 (M−H)⁻, calcd. 443.2089.

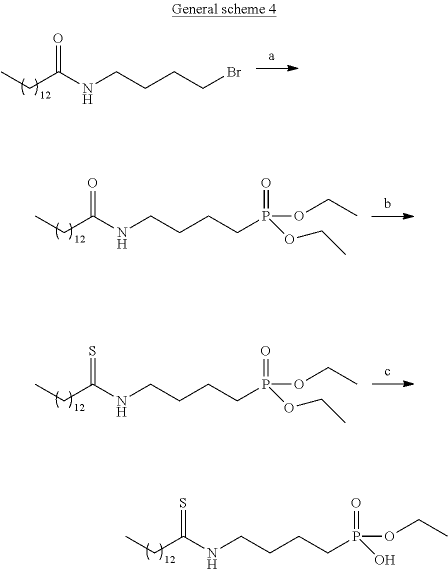

(a) P(OEt)₃, reflux; (b) Lawesson's reagent, THF; (c) 1N NaOH, EtOH.

(4a) General Procedure for the Synthesis of Phosphonates

The appropriate bromide (1.0 mmol) was added to triethyl phosphite (5 mmol, 0.9 mL) slowly at room temperature. The reaction mixture was refluxed overnight, and then excess triethyl phosphate and other volatile compounds were distilled out under reduced pressure. The residue was purified by column chromatography using CHCl₃: MeOH (95:5) as eluent.

### Diethyl 4-tetradecanamidobutylphosphonate

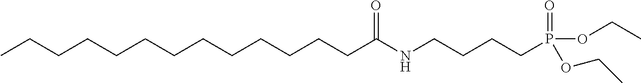 [SMILES: CCCCCCCCCCCCCC(=O)NCCCCP(=O)(OCC)OCC]

Oil, yield 60%; ¹H NMR (200 MHz, CDCl₃) δ 6.35-6.14 (m, 1H, NH), 4.17-3.81 (m, 4H, 2×CH₂), 2.06 (t, J=7.4 Hz, 2H, CH₂), 1.78-1.37 (m, 8H, 4×CH₂), 1.34-0.94 (m, 26H, 10×CH₂, 2×CH₃), 0.78 (t, J=6.6 Hz, 3H, CH₃); ¹³C NMR (50 MHz, CDCl₃) δ 173.2, 61.3 (J=6.5 Hz), 31.7, 30.2, 29.9, 29.4, 29.3, 29.2, 29.1, 24.8 (J=140.9 Hz), 25.7, 22.5, 19.7 (J=5.0 Hz), 16.3 (J=6.1 Hz), 13.9; ³¹P NMR (81 MHz, CDCl₃) δ 32.9; MS (ESI) m/z (%): 420.3 (100) [M+H]⁺.

(4b) General Procedure for the Synthesis of Thioamides

Lawesson's reagent (0.6 mmol, 243 mg) was added to a stirred solution of the amide (1.0 mmol) in dry THF (5 mL) and the reaction mixture was stirred overnight at room temperature. The organic solvent was evaporated under reduced pressure and the residue was purified by column chromatography using petroleum ether/AcOEt 8:2 as eluent.

### Diethyl 4-tetradecanethioamidobutylphosphonate

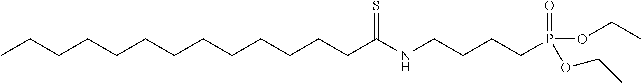 [SMILES: CCCCCCCCCCCCCC(=S)NCCCCP(=O)(OCC)OCC]

Yellow oil, yield 78%; ¹H NMR (200 MHz, CDCl₃) δ 8.47 (br, 1H, NH), 4.23-3.87 (m, 4H, 2×CH₂), 3.76-3.45 (m, 2H, CH₂), 2.72-2.45 (m, 2H, CH₂), 1.91-1.44 (m, 8H, 4×CH₂), 1.42-0.98 (m, 26H, 10×CH₂, 2×CH₃), 0.84 (t, J=6.6 Hz, 3H, CH₃); ¹³C NMR (50 MHz, CDCl₃) δ 205.5, 61.7 (J=4.0 Hz), 46.8, 45.2, 31.8, 29.5, 29.4, 29.3, 29.2, 28.9, 22.6, 19.8, 16.4 (J=5.0 Hz), 14.0; ³¹P NMR (81 MHz, CDCl₃) δ 32.9.

(4c) General Procedure for the Saponification of Diethyl Phosphonates

To a stirred solution of diethyl phosphonate (1.0 mmol) in MeOH (10 mL), aqueous NaOH 1N (1.2 mmol, 1.2 mL) was added and the reaction mixture was stirred overnight at room temperature. The organic solvent was evaporated under reduced pressure, water was added (10 mL), the mixture was acidified with HCl 1N until pH=2-3 and the aqueous solution was extracted with EtOAc (3×10 mL). The organic phase was dried over Na₂SO₄ and evaporated under reduced pressure. The residue was purified by column chromatography using CHCl₃/MeOH/AcOH 9:1:0.5 as eluent.

### Ethyl hydrogen 4-tetradecanethioamidobutylphosphonate

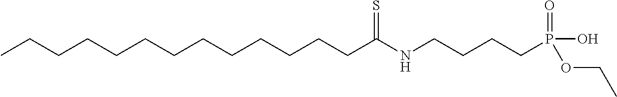 [SMILES: CCCCCCCCCCCCCC(=S)NCCCCP(=O)(O)OCC]

White solid, yield 47%; ¹H NMR (200 MHz, CDCl₃) δ 8.14 (br, 1H, NH), 4.18-3.83 (m, 2H, CH₂), 3.78-3.46 (m, 2H, CH₂), 2.77-2.49 (m, 2H, CH₂), 1.96-1.48 (m, 8H, 4×CH₂), 1.46-1.06 (m, 23H, 10×CH₂, CH₃), 0.88 (t, J=6.6 Hz, 3H, CH₃); ¹³C NMR (50 MHz, CDCl₃) δ 205.8, 61.1, 47.0, 45.5, 31.9, 29.7, 29.6, 29.4, 29.1, 22.7, 20.3, 16.5, 14.1; ³¹P NMR (81 MHz, CDCl₃) δ 31.8; HRMS (ESI) calcd for C₂₀H₄₁NO₃PS [M−H]⁻: 406.2550. Found: 406.2522.

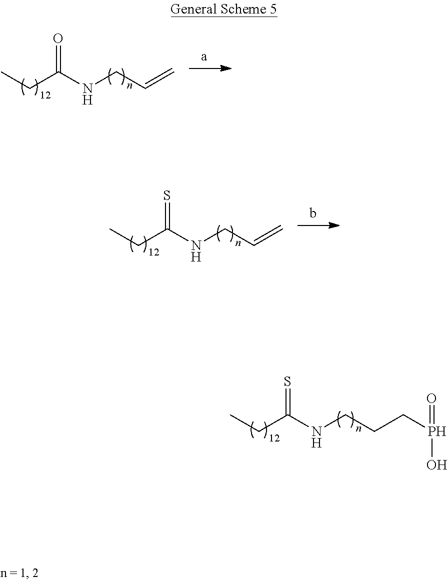

(a) Lawesson's reagent, toluene, 50° C.; (b) NH₄.H₂PO₂, AIBN, MeOH, reflux.

(5a) General Procedure for the Thionation Reaction

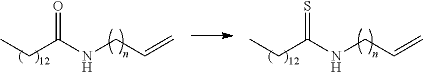

Lawesson's reagent (404 mg, 1 mmol) was added to a stirred solution of the appropriate amide (1 mmol) in toluene (5.5 mL), and the reaction mixture was warmed at 50° C. under stirring for 10 h. The solvent was removed and the product was purified by column chromatography using petroleum ether./EtOAc: 8/2 as eluent.

### N-Allyltetradecanethioamide

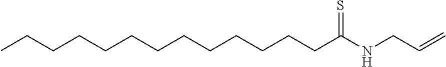 [SMILES: C=CCNC(=S)CCCCCCCCCCCCC]

Solid, yield 69%. ^{t}H NMR (200 MHz, CDCl₃) δ 7.37 (bs, 1H, NH), 6.06-5.70 (m, 1H, CH═), 5.32-5.18 (m, 2H, CH₂═), 4.35-4.22 (m, 2H, CH₂N), 2.69-2.59 (m, 2H, CH₂CS), 1.80-1.67 (m, 2H, CH₂), 1.42-1.05 (m, 20H, CH₂), 0.85 (t, J=6.3 Hz, 3H, CH₃); ¹³C NMR (50 MHz, CDCl₃) δ 206.0, 132.0, 118.6, 48.5, 47.3, 32.0, 29.7, 29.6, 29.4, 29.0, 22.8, 14.2; ESMS m/z calculated for C₁₇H₃₄NS (M+H)⁺: 284.2, found: 284.2.

### N-(But-3-en-1-yl)tetradecanethioamide

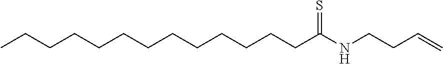 [SMILES: C=CCCNC(=S)CCCCCCCCCCCCC]

Solid, yield 72%; ¹H NMR (200 MHz, CDCl₃) δ 7.18 (bs, 1H, NH), 5.79-5.65 (m, 1H, CH═), 5.21-5.10 (m, 2H, CH₂═), 3.70-3.65 (m, 2H, CH₂N), 2.70-2.53 (m, 2H, CH₂CS), 2.41 (q, J=6.7 Hz, 2H, CH₂CH), 1.88-1.60 (m, 2H, CH₂), 1.24 (s, 20H, CH₂), 0.86 (t, J=6.4 Hz, 3H, CH₃); ¹³C NMR (50 MHz, CDCl₃) δ 205.7, 134.7, 117.8, 47.3, 44.7, 32.1, 31.9, 29.7, 29.5, 29.4, 29.4, 28.9, 22.7, 14.2. ESMS m/z calculated for C₁₈H₃₆NS (M+H)⁺: 298,2, found: 298.2.

(5b) General Procedure for the Hydrophosphinylation Reaction

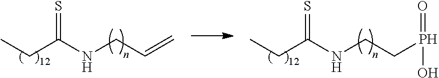

To a stirred solution of alkene (1 mmol) in MeOH (8 mL), NH₄.H₂PO₂ (330 mg, 4 mmol) and AIBN (66 mg, 0.4 mmol) were added and the reaction mixture was refluxed for 72 h. Then, the solvent was removed under reduced pressure and EtOAc (40 mL) was added. The organic phase was washed with HCl 0.5N (15 mL), H₂O (15 mL), dried over Na₂SO₄, concentrated in vacuo and the product was purified by column chromatography using a gradient of CHCl₃/MeOH/AcOH form 9/1/0.5 to 7/1/0.5.

### (3-Tetradecanethioamidopropyl)phosphinic acid

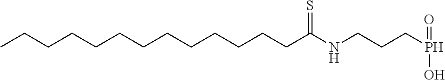

Solid, yield 55%; ¹H NMR (200 MHz, CDCl₃/DMSO: 8/2) δ 9.61 (bs, 1H, POH), 6.98 (d, J_{PH}=531.1 Hz, 1H, PH), 3.54 (bs, 2H, CH₂N), 2.12-1.48 (m, 6H, CH₂), 1.12 (s, 20H, CH₂), 0.74 (t, J=6.2 Hz, 3H); ¹³C NMR (50 MHz, CDCl₃/DMSO: 8/2) δ 210.3, 51.1, 50.8, 41.3, 36.8, 34.5, 34.4, 34.3, 34.2, 33.9, 33.6, 31.8, 30.8, 27.5, 24.4, 19.1; ³¹P NMR (81 MHz, CDCl₃/DMSO: 8/2) δ 32.84; HRMS m/z calculated for C₁₇H₃₅NO₂PS (M−H)⁻: 348.2132, found: 348.2139.

### (4-Tetradecanethioamidobutyl)phosphinic acid

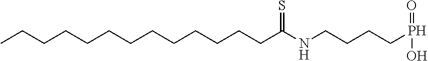

Solid, yield 33%; ¹H NMR (200 MHz, CDCl₃/DMSO: 8/2) δ 7.91 (bs, 1H, POH), 7.10 (d, J_{pH}=543.0 Hz, 1H, PH), 3.67 (bs, 2H, CH₂N), 2.63 (t, J=7.5 Hz, 2H, CH₂), 1.72 (bs, 7H, CH₂, CHP), 1.39-1.09 (m, 21H, CH₂, PH), 0.87 (t, J=6.2 Hz, 3H, CH₃); ¹³C NMR (50 MHz, CDCl₃/DMSO: 8/2) δ 206.1, 47.3, 45.4, 32.1, 29.8, 29.7, 29.6, 29.5, 29.2, 28.6, 28.3, 28.0, 25.4, 22.8, 18.3, 14.3; ³¹P NMR (81 MHz, CDCl₃/DMSO 8/2) δ 37.75; HRMS m/z calculated for C₁₈H₃₇NO₂PS (M−H)⁻: 362.2288, found: 362.2303.

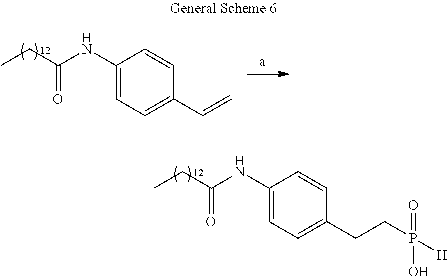

(a) H₃PO₂.NH₃, TMSCl, DIPEA, CH₂Cl₂, rt, 48 h.

### (4-Tetradecanethioamidophenethyl)phosphinic acid

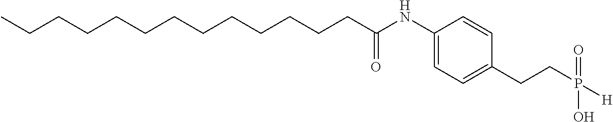 [SMILES: CCCCCCCCCCCCCC(=O)Nc1ccc(CC[PH](=O)O)cc1]

(6a) General P-Michael Hydrophosphinylation Reaction

To a stirred solution of alkene (1 mmol) in CH₂Cl₂ (10 mL), NH₄.H₂PO₂ (330 mg, 4 mmol) and DIPEA (4.1 mL, 25 mmol) were added and the reaction mixture was cooled to 0° C. under argon. TMSCl (2.95 mL, 25 mmol) was added at 0° C. and then the reaction mixture was left under stirring at room temperature for 48 hours. MeOH (2 mL) was added and left for 15 min followed by concentration under reduced pressure. EtOAc (40 mL) was added to the residue and the organic phase was washed with HCl 0.5N (15 mL), H₂O (15 mL), dried over Na₂SO₄, concentrated in vacuo and the crude product was purified by column chromatography using a gradient of CHCl₃/MeOH/AcOH form 9/1/0.5 to 7/1/0.5.

Solid, yield 35%; ¹H NMR (200 MHz, CDCl₃/DMSO: 8/2) δ 10.31 (s, 1H, POH), 7.42 (d, J=8.6 Hz, 2H, arom), 7.00 (d, J=8.5 Hz, 2H, arom), 6.97 (d, J_{PH}=528.2 Hz, 1H, PH), 2.71 (bs, 2H, CH₂CON), 2.21 (t, J=7.4 Hz, 2H, CH₂Ph), 1.60 (m, 2H, CH₂), 1.22-1.05 (s, 22H, CH₂), 0.75 (t, J=5.8 Hz, 3H, CH₃); ¹³C NMR (50 MHz, CDCl₃/DMSO: 8/2) δ 173.8 142.1, 140.4, 128.3, 120.1, 54.0, 44.0, 37.4, 32.0, 29.8, 29.5, 22.8, 18.7, 17.4, 14.4, 12.4; ³¹P NMR (81 MHz, CDCl₃/DMSO: 8/2) δ 27.39; HRMS m/z calculated for C₂₂H₃₇NO₃P (M−H)⁻: 394.2517, found: 394.2512.

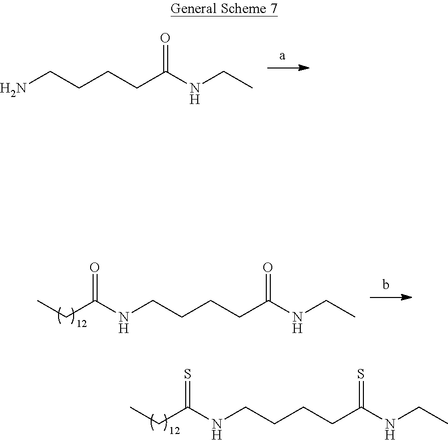

(a) myristic acid, WSCI.HCl, HOBt, Et₃N, CH₂Cl₂; (b) Lawesson's reagent THF.

### N-(5-(Ethylamino)-5-oxopentyl)tetradecanamide

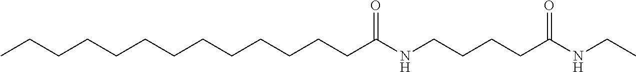 [SMILES: CCCCCCCCCCCCCC(=O)NCCCCC(=O)NCC]

To a stirred solution of the amine (1.0 mmol) in CH₂Cl₂ (10 mL) at 0° C., Et₃N (0.15 mL, 1.1 mmol) was added, and subsequently myristic acid (1.0 mmol), WSCI (0.21 g, 1.1 mmol) and HOBt (0.14 g, 1.0 mmol) were added at 0° C. The reaction mixture was stirred for 1 h at 0° C. and overnight at room temperature. The solvent was evaporated under reduced pressure and EtOAc (20 mL) was added. The organic layer was washed consecutively with brine, 1 N HCl, brine, 5% NaHCO₃, and brine, dried over Na₂SO₄ and evaporated under reduced pressure. The residue was purified by column chromatography using CHCl₃/MeOH 98:2 as eluent.

White solid, yield 58%; ¹HNMR (200 MHz, CDCl₃/CD₃OD) δ 3.17-2.96 (m, 4H, 2×CH₂), 2.10-1.94 (m, 4H, 2×CH₂), 1.57-1.26 (m, 6H, 3×CH₂), 1.24-1.05 (m, 20H, 10×CH₂), 0.99 (t, J=7.3 Hz, 3H, CH₃), 0.74 (t, J=6.8 Hz, 3H, CH₃); ¹³C NMR (50 MHz, CDCl₃/CD₃OD) δ 174.9, 174.2, 38.6, 36.7, 35.5, 34.4, 32.0, 29.8, 29.6, 29.5, 29.4, 28.5, 26.0, 22.8, 14.5, 14.2.

### N-(5-(Ethylamino)-5-thioxopentyl)tetradecanethioamide

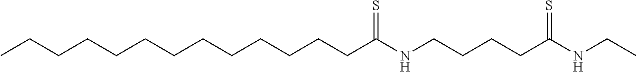 [SMILES: CCCCCCCCCCCCCC(=S)NCCCCC(=S)NCC]

To a stirred solution of the amide (1.0 mmol) in dry THF (5 mL) Lawesson's reagent (1.2 mmol, 485 mg) was added and the reaction mixture was stirred overnight at room temperature. The organic solvent was evaporated under reduced pressure and the residue was purified by column chromatography using PE/AcOEt 8:2 as eluent.

White solid, yield 63%; ¹H NMR (200 MHz, CDCl₃) δ 8.01 (br s, 1H, NH), 7.87 (br s, 1H, NH), 3.78-3.50 (m, 4H, 2×CH₂), 2.78-2.50 (m, 4H, 2×CH₂), 1.95-1.54 (m, 6H, 3×CH₂), 1.40-1.03 (m, 27H, 12×CH₂, CH₃), 0.85 (t, J=6.6 Hz, CH₃); ¹³C NMR (50 MHz, CDCl₃) δ 205.7, 204.1, 47.1, 45.1, 45.0, 41.0, 31.8, 29.5, 29.4, 29.3, 28.9, 26.4, 25.8, 22.6, 14.0, 13.0; HRMS (ESI) calcd for C₂₁H₄₁N₂S₂ [M−H]⁻: 385.2717. Found: 385.2721.

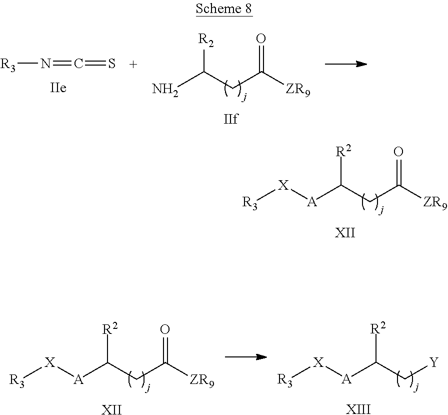

### 5-(3-Dodecylthioureido)-N-ethylpentanamide

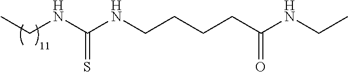

To a stirred solution of the amine (1.0 mmol) in CH₂Cl₂ (10 mL) the isothiocyanate (1.1 mmol, 250 mg) was added, and the reaction mixture was stirred overnight at room temperature. The solvent was evaporated under reduced pressure and the product was obtained by treatment with ether/petroleum ether.

White solid. Yield 46%; ¹H NMR (CDCl₃) δ 6.49 (br, 1H, NH), 6.15 (br, 1H, NH), 5.76 (br, 1H, NH), 3.72-3.14 (m, 6H, 3×CH₂), 2.23 (t, J=6.6 Hz, 2H, CH₂), 1.80-1.49 (m, 6H, 3×CH₂), 1.43-1.19 (m, 18H, 9×CH₂), 1.15 (t, J=7.2 Hz, 3H, CH₃), 0.88 (t, J=6.6 Hz, 3H, CH₃); ¹³C NMR (CDCl₃) δ 181.2, 173.3, 44.1, 43.4, 35.4, 34.1, 31.6, 29.3, 28.1, 26.7, 22.4, 14.3, 13.8.

### 5-(3-Dodecylthioureido)-N-ethylpentanethioamide

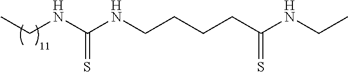

White solid. Yield 64%; ¹H NMR (CDCl₃) δ 8.17 (br, 1H, NH), 6.49-6.11 (br, 2H, 2×NH), 3.78-3.22 (m, 6H, 3×CH₂), 2.68 (t, J=7.2 Hz, 2H, CH₂), 1.91-1.71 (m, 2H, CH₂), 1.70-1.44 (m, 4H, 2×CH₂), 1.42-1.13 (m, 21H, 9×CH₂, CH₃), 0.86 (t, J=6.6 Hz, 3H, CH₃); ¹³C NMR (CDCl₃) δ 204.2, 181.1, 45.4, 44.3, 43.7, 41.0, 31.8, 29.5, 29.2, 28.9, 28.0, 26.9, 26.8, 25.9, 22.6, 14.1, 13.0; HRMS (ESI) calcd for C₂₀H₄₀N₃S₂ [M−H]⁻: 386.2669. Found: 386.2654

### Methyl (S)-5-(3-(4-oxo-4-(phenylamino)butyl)thioureido)nonanoate

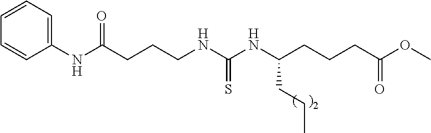

Oil. Yield 40%. ¹H NMR (CDCl₃) δ 9.02 (s, 1H, NH), 7.49 (d, J=8.0 Hz, 2H, arom), 7.23 (t, J=8.0 Hz, 2H, arom), 7.02 (t, J=8.0 Hz, 1H, arom), 6.86 (s, 1H, NHCS), 6.29 (s, 1H, NHCS), 3.57 (s, 3H, OCH₃), 3.50-3.40 (m, 1H, CH), 3.20-2.80 (m, 2H, CH₂), 2.50-2.30 (m, 2H, CH₂), 2.30-2.10 (m, 2H, CH₂), 2.00-1.70 (m, 2H, CH₂), 1.70-1.00 (m, 10H, CH₂), 0.79 (t, J=6.0 Hz, 3H, CH₃); ¹³C NMR (CDCl₃) δ 180.8, 174.3, 172.1, 138.0, 128.7, 124.0, 119.9, 53.8, 51.5, 38.8, 36.2, 34.3, 33.9, 33.6, 27.7, 24.7, 22.5, 20.9, 13.8; MS (ESI) m/z (%) 408.33 [(M+H)⁺, 100].

### (S)-N-Hydroxy-5-(3-(4-oxo-4-(phenylamino)butyl)thioureido)nonamide

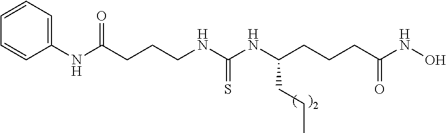

Off-white solid. Yield 5%. ¹H NMR (CDCl₃/CD₃OD) δ 7.50 (d, J=8.0 Hz, 2H, arom), 7.23 (t, J=8.0 Hz, 2H, arom), 7.01 (t, J=8.0 Hz, 1H, arom), 3.70-3.40 (m, 1H, CH), 2.32 (t, J=8.0 Hz, 2H, CH₂), 2.10-1.90 (m, 2H, CH₂), 1.90-1.60 (m, 2H, CH₂), 1.60-1.00 (m, 12H, CH₂), 0.80 (t, J=6.0 Hz, 3H, CH₃); ¹³C NMR (CDCl₃/CD₃OD) δ 181.0, 173.0, 171.2, 134.0, 128.3, 123.2, 119.8, 48.6, 39.1, 35.6, 32.6, 30.0, 29.7, 28.3, 27.2, 24.4, 22.8, 14.2; MS (ESI) m/z (%) 409.30 [(M+H)⁺, 100].

### Example 2

### Inhibitory Activities

The in vitro activity of the synthetic inhibitors was studied using the Amplex™ Red PLD assay kit (Molecular Probes, Interchim, Montlucon, France). Inhibitors were dissolved in DMSO (stock solution 3 mM) and appropriate dilutions were made in DMSO. The inhibition of ATX by the synthetic inhibitors was measured employing 2 nM recombinant ATX (mouse ATX-β from Sino Biological), 50 μM LPC (16:0, 18:0 or 18:1) and 0.001 to 5 μmol/L of inhibitors (final concentrations). The IC₅₀ values were determined using the mean values obtained from two independent experiments. The IC₅₀ values were determined graphically using the sigmoid dose-response fitting method (PrismH software) by the equation f=y0+a/(1+exp(−(x−x0)/b)) given by SigmaPlot 11.0.

TABLE 1
Inhibition of ATX by various hydroxamates inhibitor compounds described herein.

| | | IC₅₀ | IC₅₀ |
|---|---|---|---|
| | | (μM) | (μM) |
| | | 16:0 | 18:0 |
| Code | Structure | LPC | LPC |
| GK337 | 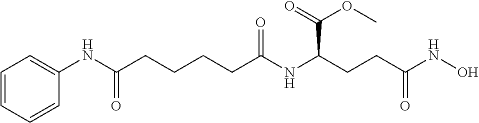 [SMILES: COC(=O)[C@@H](CCC(=O)NO)NC(=O)CCCCC(=O)Nc1ccccc1] | 0.4 | 5 |
| GK382 | 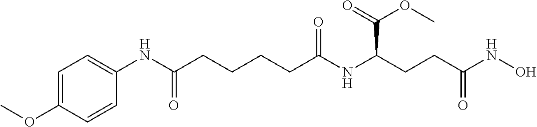 [SMILES: COC(=O)[C@@H](CCC(=O)NO)NC(=O)CCCCC(=O)Nc1ccc(OC)cc1] | 4 | |
| GK400 | 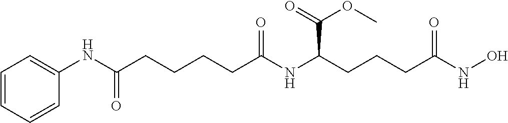 [SMILES: COC(=O)[C@@H](CCCC(=O)NO)NC(=O)CCCCC(=O)Nc1ccccc1] | 0.05 | |
| GK332 | 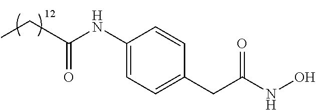 | 1 | 0.2 |
| GK345 | 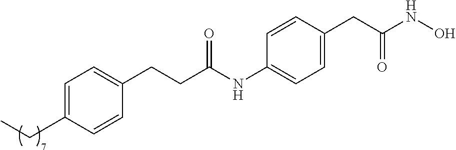 | 5 | |
| GK344 | 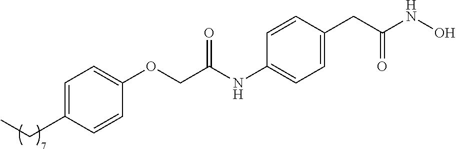 | 1 | 0.3 |
| GK341 | 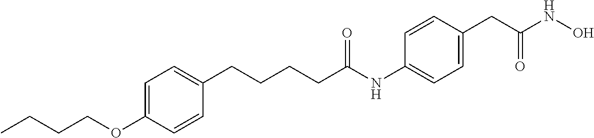 [SMILES: CCCCOc1ccc(CCCCC(=O)Nc2ccc(CC(=O)NO)cc2)cc1] | 4 | |
| GK285 | 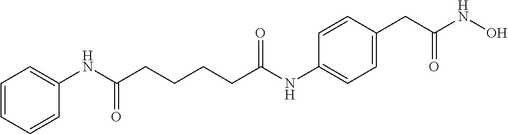 [SMILES: O=C(Cc1ccc(NC(=O)CCCCC(=O)Nc2ccccc2)cc1)NO] | 1.2 | |
| GK346 | 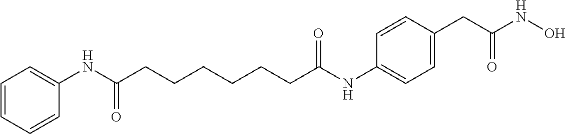 [SMILES: O=C(Cc1ccc(NC(=O)CCCCCCC(=O)Nc2ccccc2)cc1)NO] | 2 | |
| GK340 | 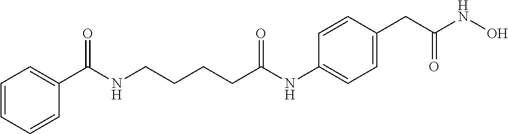 [SMILES: O=C(Cc1ccc(NC(=O)CCCCNC(=O)c2ccccc2)cc1)NO] | 1 | 0.2 |
| GK381 | 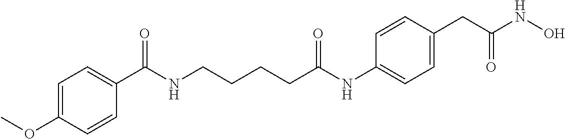 [SMILES: COc1ccc(C(=O)NCCCCC(=O)Nc2ccc(CC(=O)NO)cc2)cc1] | 1 | |
| GK333 | 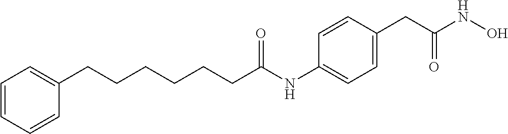 [SMILES: O=C(Cc1ccc(NC(=O)CCCCCCc2ccccc2)cc1)NO] | 0.7 | 2 |
| GK335 | 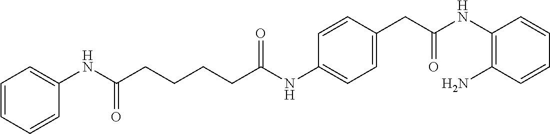 [SMILES: Nc1ccccc1NC(=O)Cc1ccc(NC(=O)CCCCC(=O)Nc2ccccc2)cc1] | 0.6 | |
| GK442 | 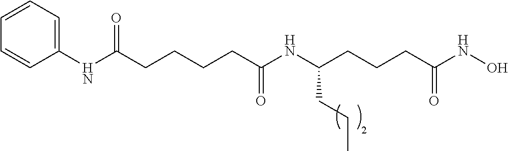 | 0.06 | |
| GK471 | 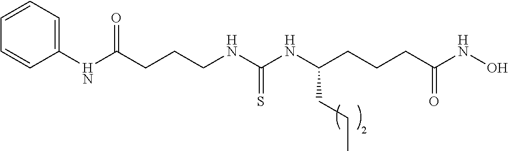 | 0.8 | |

TABLE 2
Inhibition of ATX by various thioamides/phosphonates/phosphinates inhibitor compounds described herein.

| | | IC₅₀ | IC₅₀ | IC₅₀ |
|---|---|---|---|---|
| | | (μM) | (μM) | (μM) |
| | | 16:0 | 18:0 | 18:1 |
| Code | Structure | LPC | LPC | LPC |
| GK378 | 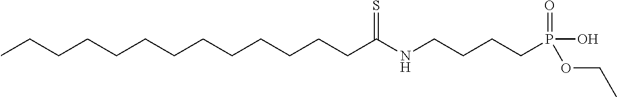 [SMILES: CCCCCCCCCCCCCC(=S)NCCCCP(=O)(O)OCC] | 0.12 | | 0.05 |
| GK312 | 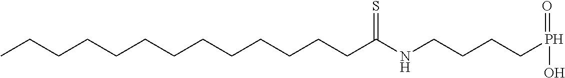 | 3 | 0.7 | |
| GK325 | 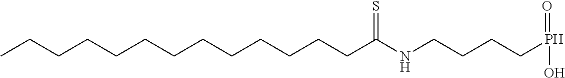 | 0.5 | 1 | |
| GK327 | 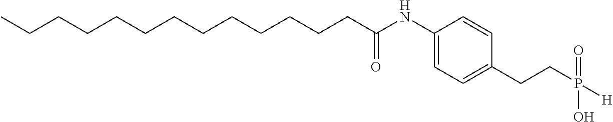 [SMILES: CCCCCCCCCCCCCC(=O)Nc1ccc(CC[PH](=O)O)cc1] | 0.4 | 4 | |
| GK377 | 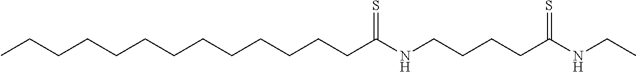 [SMILES: CCCCCCCCCCCCCC(=S)NCCCCC(=S)NCC] | 0.198 | | 0.098 |
| GK446 | 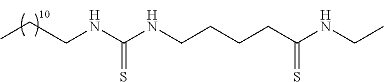 | 0.06 | | |

Although the present invention has been described hereinabove by way of specific embodiments thereof, it can be modified, without departing from the spirit and nature of the subject invention as defined in the appended claims.

1. A compound of formula I 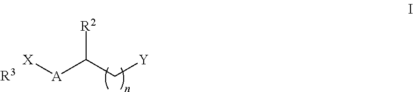
wherein:
A represents 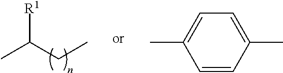
X, Y: groups able to form complexes with Zn ion,
wherein
Y: 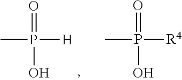
R⁴: lower alkyl, lower alkoxy 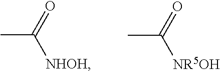
R⁵: phenyl, benzyl, lower alkyl 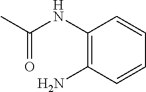 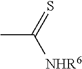
R⁶: lower alkyl, CH₂CH₂NH₂
X: 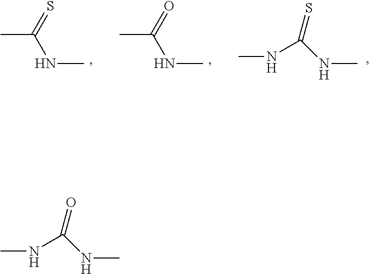
R¹, R² is independently selected from H, lower alkyl, COOR⁷, CONHR⁷, CSNHR⁷, wherein R⁷ is a lower alkyl group
R³: C₆-C₁₆ (alkyl, alkenyl, alkynyl) or a group selected from 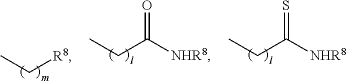
R⁸ : aryl, heteroaryl
k=0-2
n=0-3
m, l=3-8
or isomers, enantiomeric forms, pharmacologically acceptable salts, or prodrugs thereof.

2. A compound of formula I as claimed in claim 1, wherein said compound is:

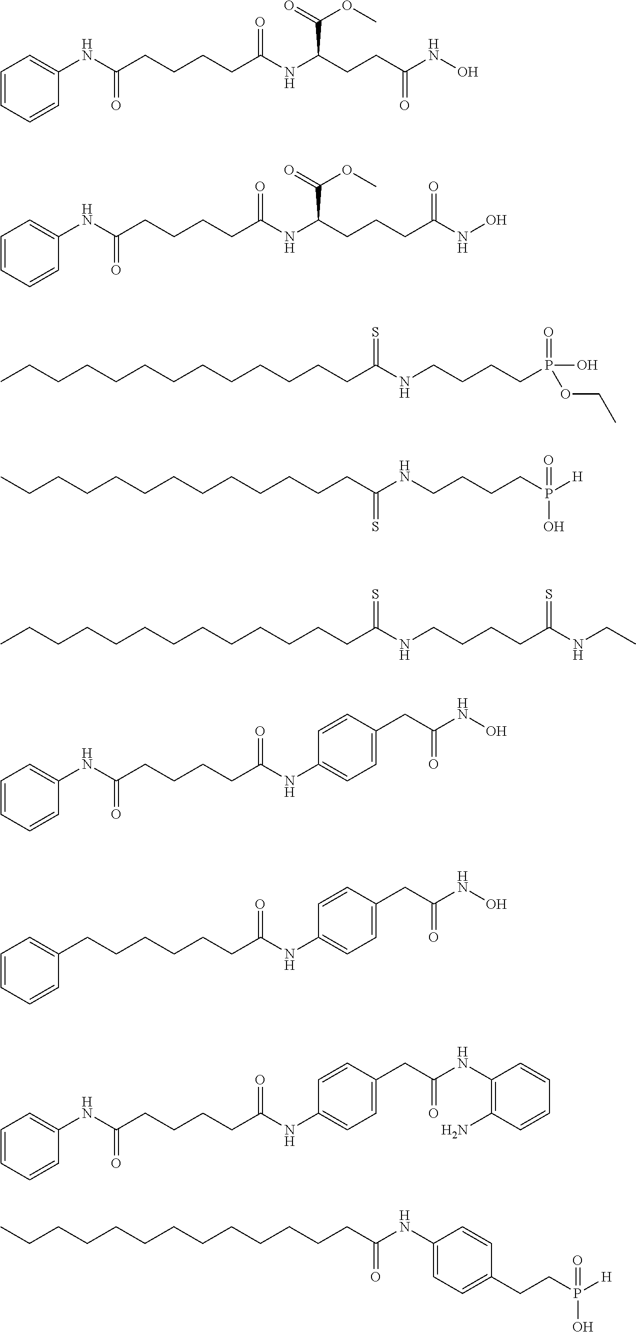

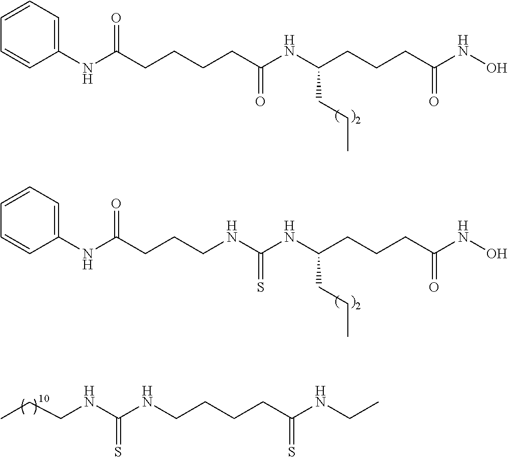

3. A compound of formula I as claimed in claims 1, wherein said compound is:

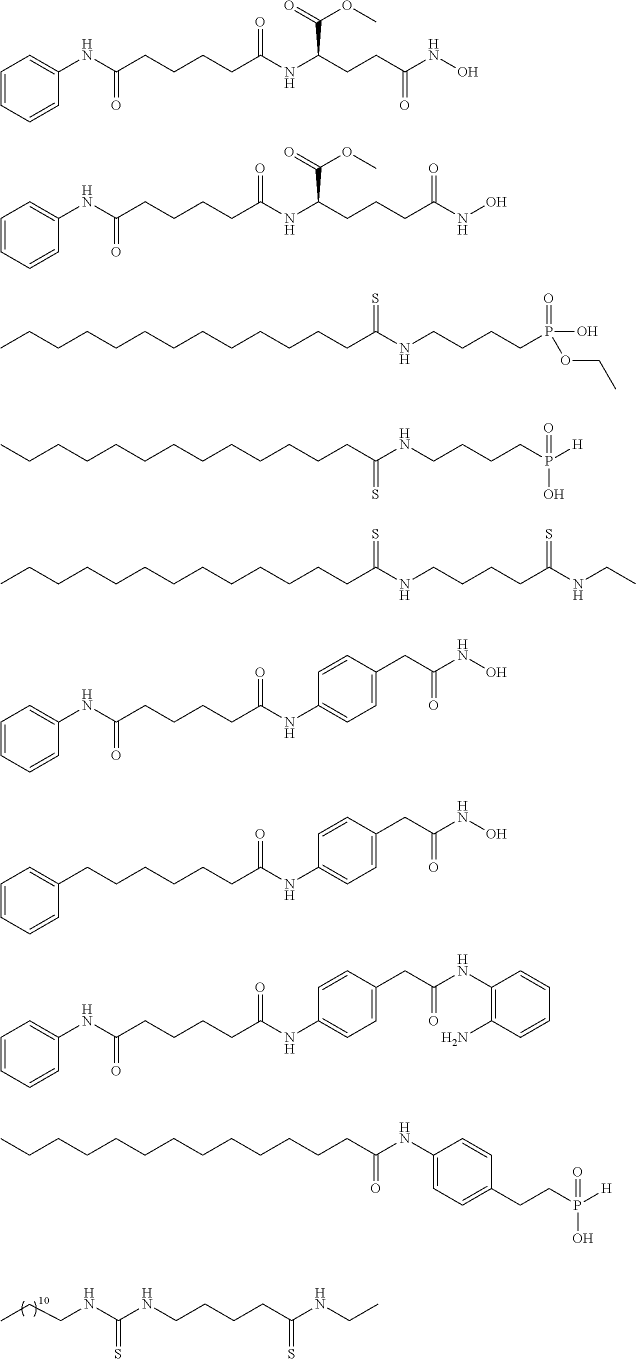

4. A compound of formula I as claimed in claim 1, wherein said compound is:

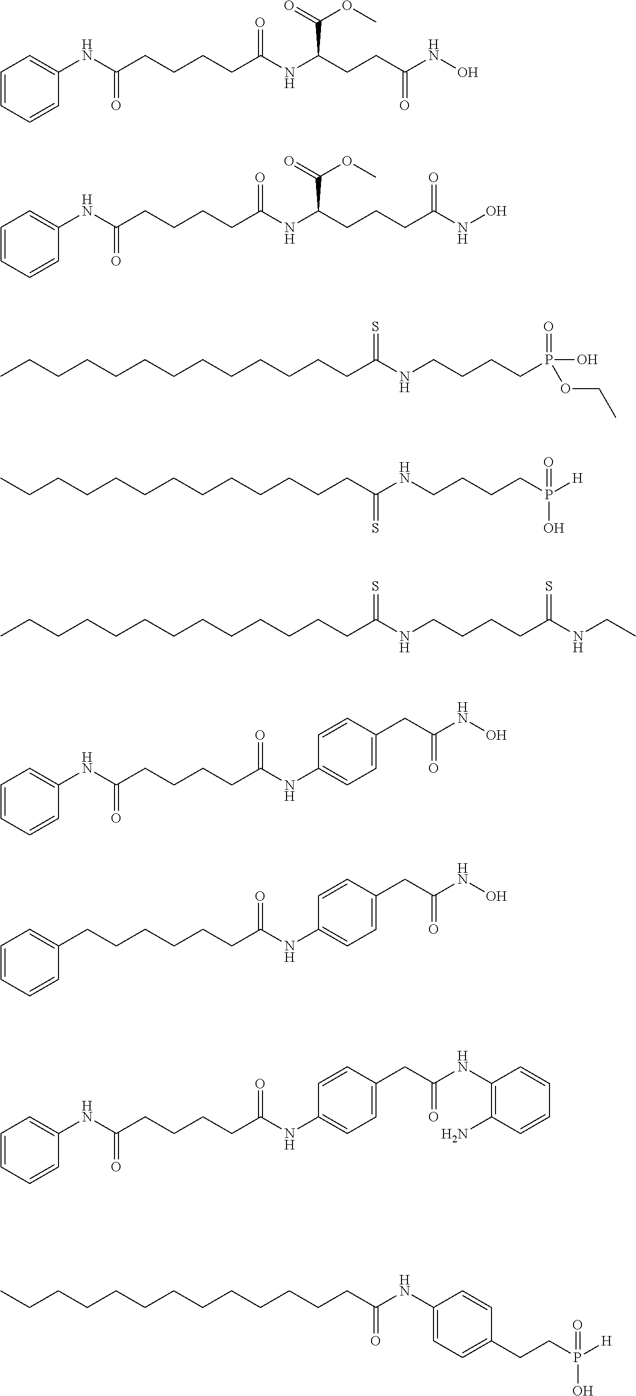

5. A composition comprising a compound according to claim 1 and a pharmaceutically acceptable carrier or excipient.

6. A compound according to claim 1 for use as a medicament.

7. A compound according to claim 1 for use in inhibiting autotaxin (ATX) activity in a biological system.

8. A compound according to claim 1 for use in the prevention or treatment of an inflammatory disease or condition in a subject.

9. A compound according to claim 1 for use in the prevention or treatment of cancer in a subject.

10. A compound according to claim 1 for use in the prevention or treatment of obesity in a subject.

11. A compound according to claim 1 for use in the prevention or treatment of autoimmune diseases in a subject.

12. A process for preparing a compound according to claim 1, comprising a step of: 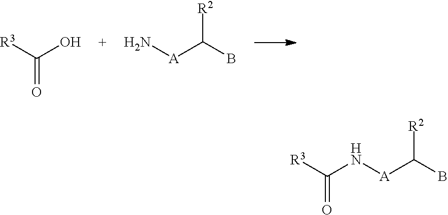
wherein
A represents 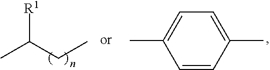
R¹, R² is independently selected from H, lower alkyl, COOR⁷, CONHR⁷, CSNHR⁷, wherein R⁷ is a lower alkyl group
R³: C₆-C₁₆ (alkyl, alkenyl, alkynyl) or a group selected from 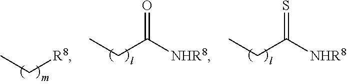
wherein R represents a aryl or heteroaryl group
B represents —(CH₂)ⱼCOOR⁹, (CH₂)ⱼCONR⁵OH —(CH₂)ⱼCONHR⁶, —(CH₂)ⱼX, —(CH₂)ⱼCH═CH₂, wherein R⁵ represents a phenyl, benzyl, or lower alkyl group, R⁶ is a lower alkyl group, CH₂CH₂NH₂, R⁹ is a lower alkyl group, and X is a halogen
m, l=3-8,
n, j=0-3.

13. A process according to claim 12, wherein B is —(CH₂)ⱼCOOR⁹, wherein R⁹ is a lower alkyl group, j=0-3, and further comprising a step of converting an ester of formula (IIa) into a hydroxamate of formula (III).

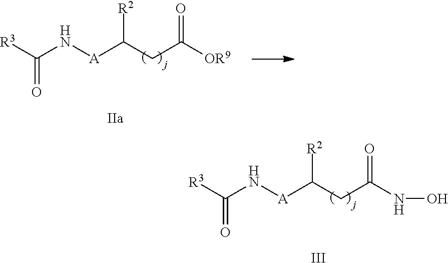

14. A process according to claim 12, wherein B is —(CH ₂)ⱼCOOR⁹, wherein R⁹ is a lower alkyl group, j=0-3), and further comprising:
(i) hydrolyzing a compound of formula (IIa) to provide the corresponding carboxylic acid of formula (IV); 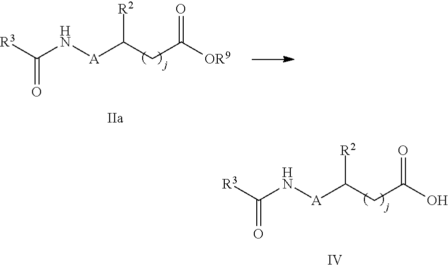
(ii) coupling the compound of formula (IV) with 1,2-phenylenediamine to provide a compound of formula (V).

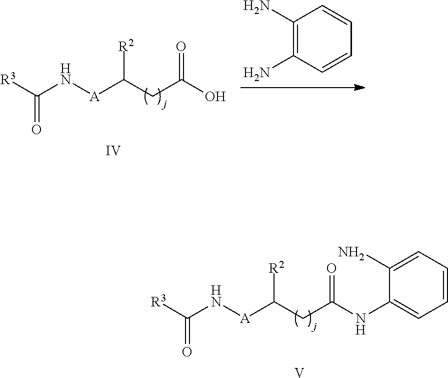

15. A process according to claim 12, wherein B is —(CH₂)ⱼX, X=halogen, j=0-3, and further comprising:
(1) Converting a compound of formula (IIb) into a compound of formula (VI); 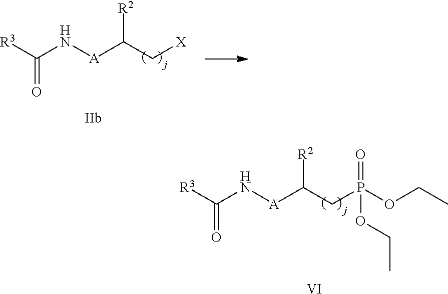
(2) Converting the compound of formula (VI) into a compound of formula (VII); 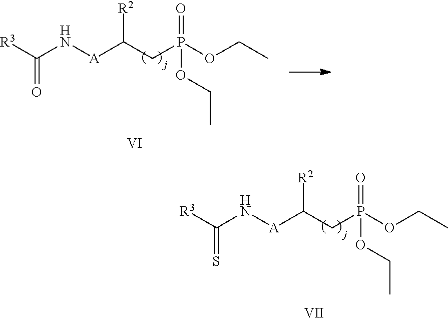
(3) Converting the compound of formula (VII) into a compound of formula (VIII).

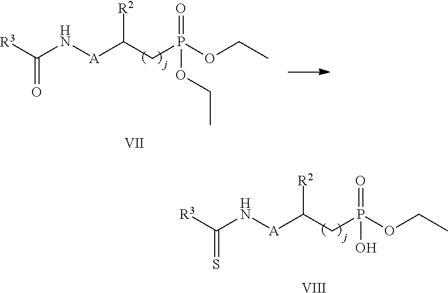

16. A process according to claim 12, wherein B is —(CH₂)ⱼCH═CH₂, j=0-3, and further comprising:
(a) Converting a compound of formula (IIc) into a compound of formula (IX); 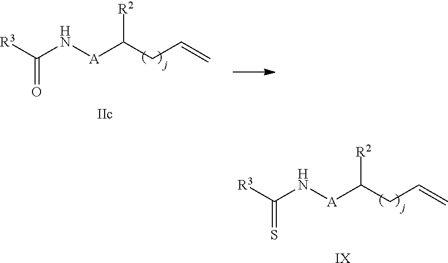
(b) Converting the compound of formula (IX) into a compound of formula (X).

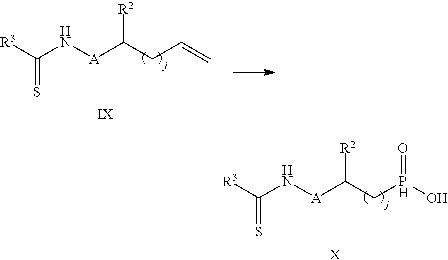

17. A process according to claim 12, wherein B is —(CH₂)ⱼCONHR⁶, wherein R⁶ is a lower alkyl group, CH₂CH₂NH₂, j=0-3, and further comprising:
(1) Converting a compound of formula (IId) into a compound of formula (XI); 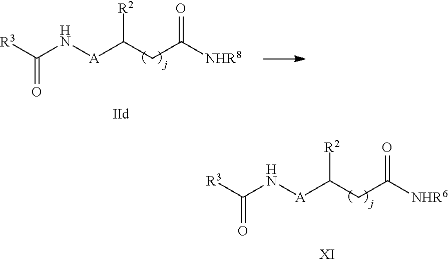
(2) Converting the compound of formula (XI) into a compound of formula (XII).

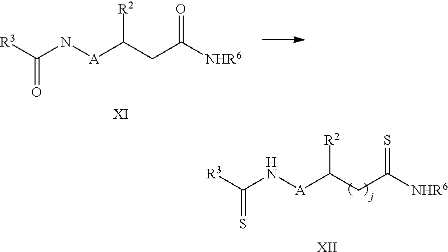

18. A process according to claim 12, wherein B is —(CH₂)ⱼCONHR⁹, wherein R⁹ is a C₁-C₈ alkyl group, CH₂CH₂NH₂, j=0-3, and further comprising:
a) Reacting a compound of formula (IIe) with a compound of formula (IIf) to provide a compound of formula (XII), wherein Z is O or N, X and Y have the same definitions as above; 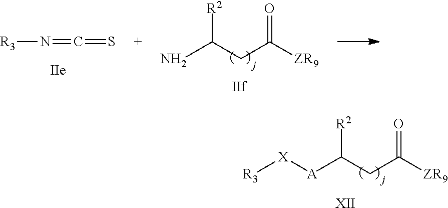
b) Converting the compound of formula (XII) into a compound of formula (XIII). [chemical structure]
